(19) United States
(12) Patent Application Publication
(10) Pub. No.: US 2009/0291428 A1
(43) Pub. Date: Nov. 26, 2009

(54) COMPOSITIONS AND METHODS FOR THE DETECTION AND TREATMENT OF POXVIRAL INFECTIONS

(75) Inventors: John P. Atkinson, St. Louis, MO (US); M. Kathryn Liszewski, St. Louis, MO (US); Marilyn K. Leung, St. Louis, MO (US); Paula Bertram, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: The Washington University, St. Louis, MO (US)

(21) Appl. No.: 12/469,535

(22) Filed: May 20, 2009

Related U.S. Application Data
(60) Provisional application No. 61/054,596, filed on May 20, 2008.

(51) Int. Cl. C12Q 1/70; C07K 16/00; C12N 7/06

(52) U.S. Cl. 435/5; 530/390.1; 530/388.3; 435/238

(57) ABSTRACT

The invention encompasses an antibody that binds to and substantially inhibits the activity of at least one poxvirus complement inhibitor. Additionally, the application encompasses methods of detecting a poxvirus complement inhibitor and methods of decreasing the activity of a poxvirus complement inhibitor.

### CROSS REFERENCE TO RELATED APPLICATIONS

This application claims the priority of U.S. provisional application No. 61/054,596, filed May 20, 2008, hereby incorporated by reference in its entirety.

### GOVERNMENTAL RIGHTS

This invention was made with government support under U54 AIO57160 awarded by the NIH. The government has certain rights in the invention.

### FIELD OF THE INVENTION

The invention encompasses an antibody that binds to and substantially inhibits the activity of at least one poxvirus complement inhibitor.

### BACKGROUND OF THE INVENTION

To establish infection, poxviruses must first subvert the innate immune response of the host. The complement system lies at the interface between innate and adaptive immunity, providing a first line of defense against a variety of pathogens. It consists of a family of soluble and cell-surface proteins that recognize pathogen-associated molecular patterns, altered-self ligands, and immune complexes. Not surprisingly, one strategy used by pathogens, including poxviruses, to control complement activation is the expression of inhibitors of complement enzymes (PICES) that mimic the host's complement regulators and thus serve as virulence factors.

The development of improved diagnostics and therapeutics to treat smallpox or other poxviral diseases is an emerging priority. Poxviral complement regulators (also called poxviral inhibitors of complement enzymes or PICES) are attractive targets for therapeutic intervention. For example, the vaccinia virus complement regulator VCP can inhibit antibody-dependent, complement-enhanced neutralization of vaccinia virus virions and viruses lacking VCP are attenuated. These studies demonstrate the attractiveness of PICES as targets to treat poxviral infections.

Despite this, however, a convenient means to inhibit PICES has not yet been developed. Hence there is a need in the art for a compound or method for inhibiting the activity of PICES.

### SUMMARY OF THE INVENTION

One aspect of the present invention encompasses an antibody that binds to and substantially inhibits the activity of at least one poxvirus complement inhibitor.

Another aspect of the invention encompasses a method for decreasing the activity of a poxvirus complement inhibitor. The method typically comprises contacting the poxvirus complement inhibitor with an antibody that binds to and substantially inhibits the activity of at least one poxvirus complement inhibitor.

Still another aspect of the invention encompasses a method for detecting a poxvirus complement inhibitor in a sample. The method comprises contacting the sample with an antibody that binds to and substantially inhibits the activity of at least one poxvirus complement inhibitor. Association between the antibody and the poxvirus complement inhibitor is then detected.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

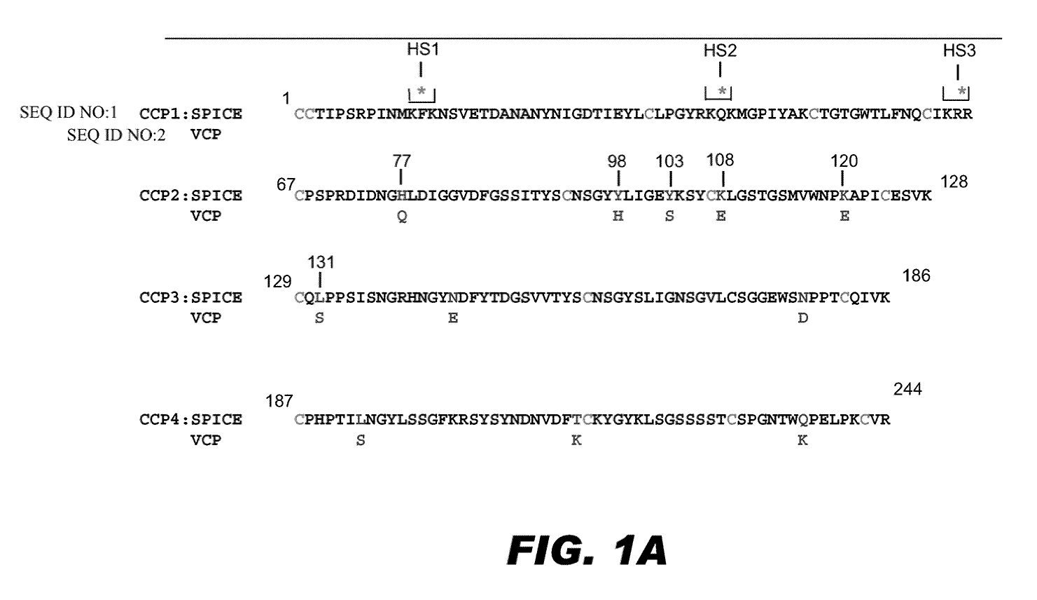

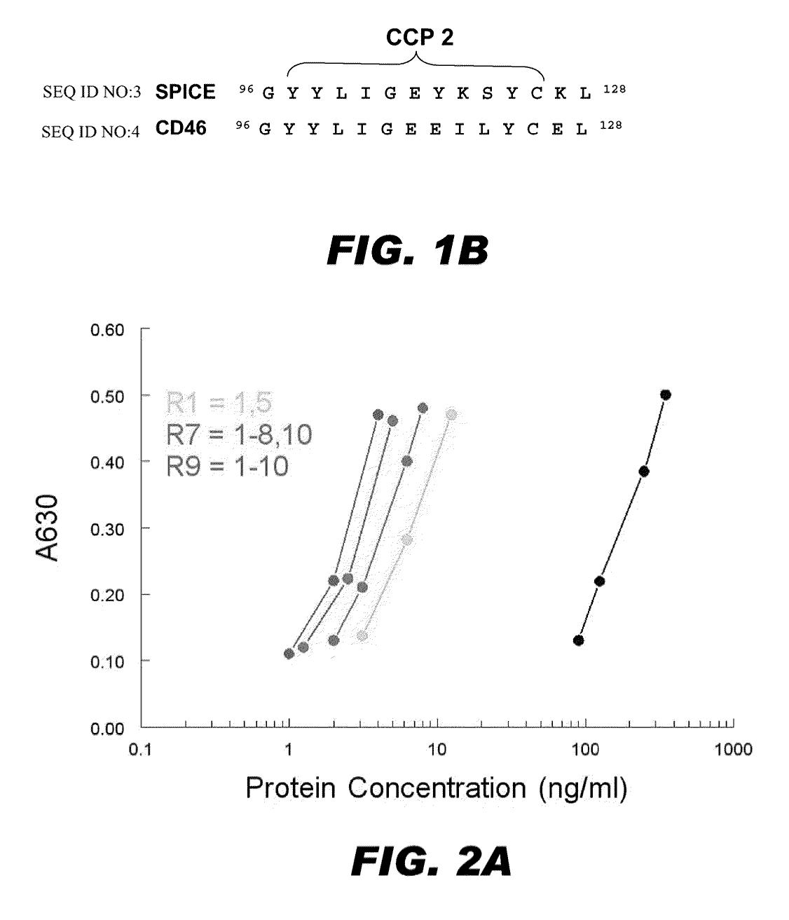

FIG. 1 depicts an alignment of SPICE and VCP and SPICE and CD46. (A) The 11 amino acid differences between the SPICE and VCP mature proteins are highlighted (blue residues). Red, cysteine residues; boxed areas, putative heparin binding sites. There are three potential heparin binding clusters in CCP1 of SPICE (HS1, HS2, HS3). Amino acid numbering conforms to those of the mature secreted protein (does not include signal peptide). (B) Alignment of residues 96-128 from CCP2 of SPICE and CD46 and description of SPICE-VCP chimeras.

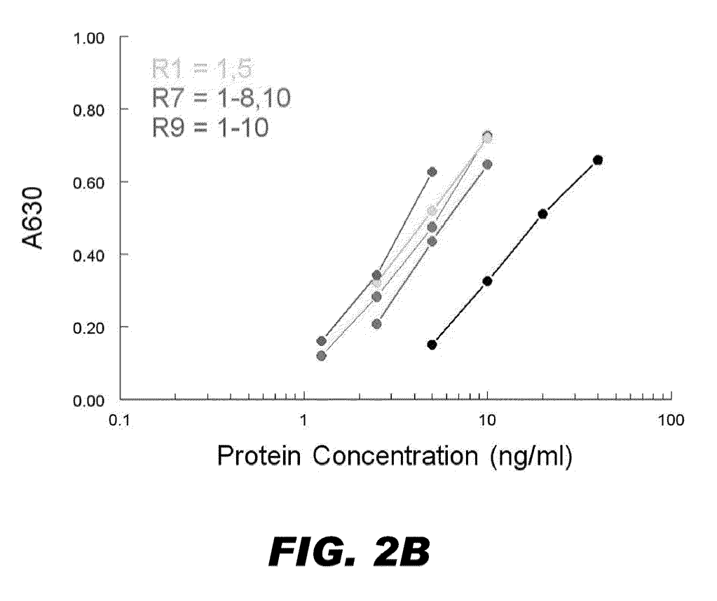

FIG. 2 depicts graphs of (A) C3b or (B) C4b binding to different SPICE-VCP chimeras. (A) VCP constructs with SPICE amino acid substitutions (see Table 1) were transiently expressed in Chinese hamster ovary (CHO) cells. Supernatants were profiled in a C3b binding ELISA. (B) C4b binding of the same SPICE-VCP chimeras. Representative experiment of three.

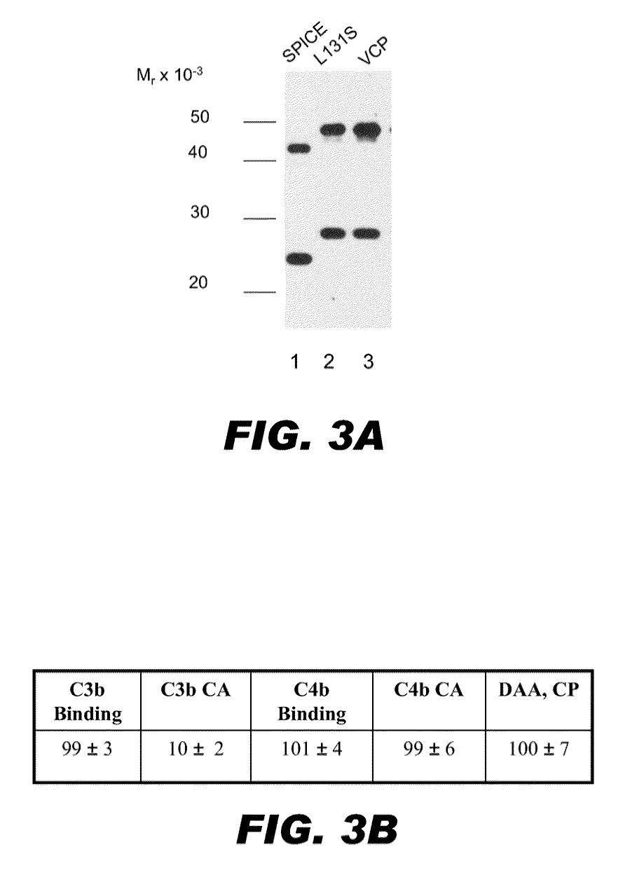

FIG. 3 depicts a western blot and functional data showing that SPICE residue L131 influences structure, function, and electrophoretic mobility. (A) Western blot of SPICE substituted with homologous serine residue of VCP at 131 (i.e., L131S) (10% SDS-PAGE). SPICE containing the single VCP residue (L131S) (lane 2) has an identical Mᵣ to that of VCP (lane 3). (B) Functional analyses of SPICE-L131S. Assays were performed for ligand binding (100 ng/ml), cofactor activity (CA) for C3b and C4b (30 pM), and classical pathway decay accelerating activity (DAA) (25 ng). Three experiments, mean+SEM.

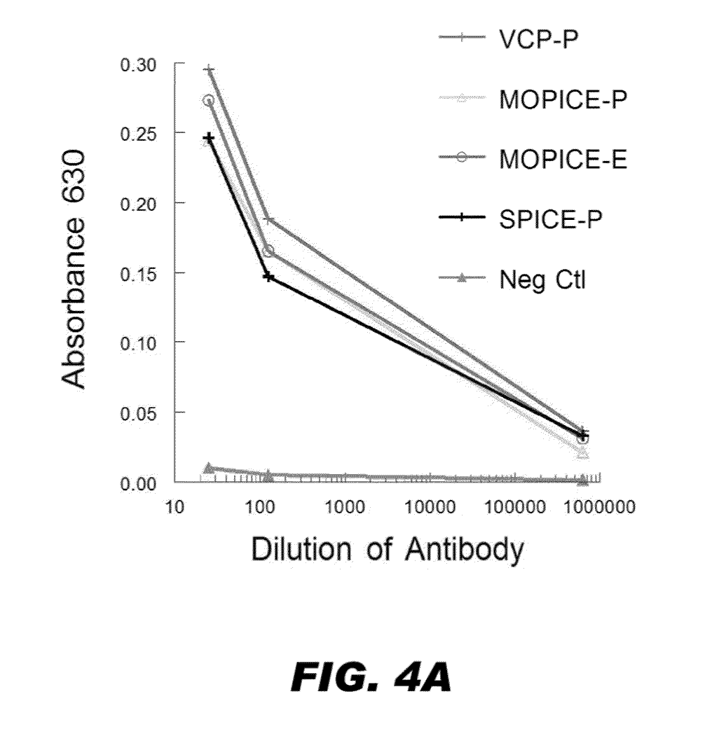

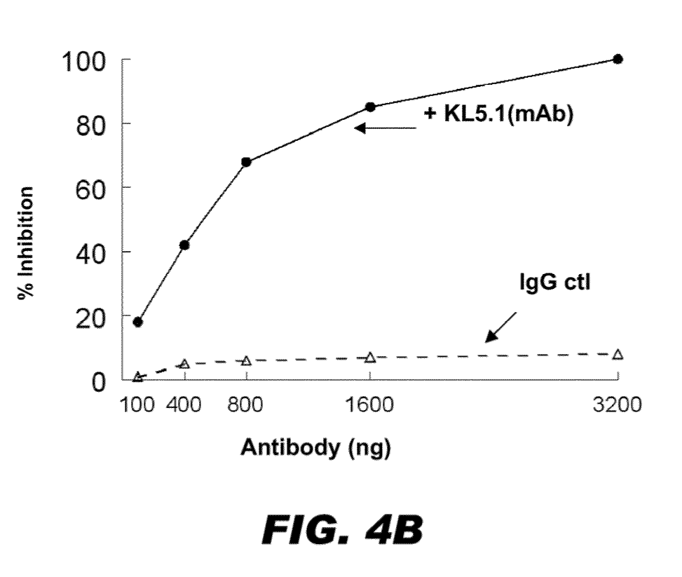

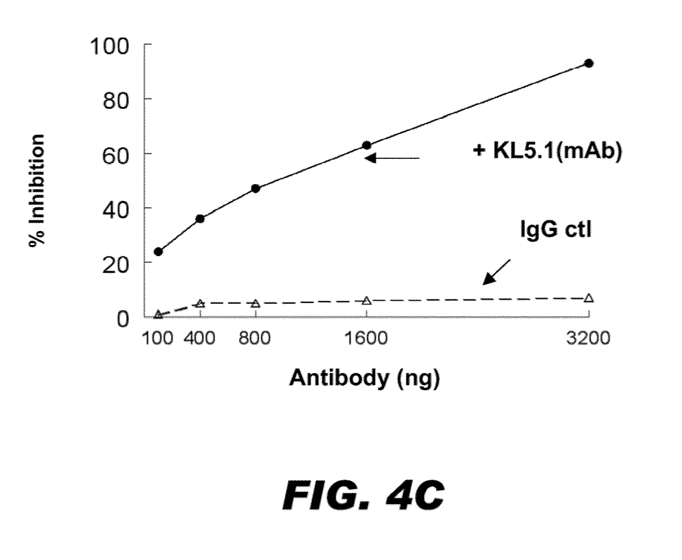

FIG. 4 depicts graphs showing binding activity and inhibition data for mAb KL5.1. (A) Anti-PICE mAb KL5.1 binds to SPICE, MOPICE and VCP. The PICE proteins were adsorbed to a microtiter plate and dilutions of Ab added followed by detection with an HRP-linked polyclonal anti-mouse gamma globulin. “P” or “E” designations refer to whether the protein was produced in Pichia pastoris or E. coli expression systems, respectively. Representative experiment of three. Monoclonal Ab KL5.1 inhibits classical pathway C3 convertase decay accelerating activity (DAA) of (B) SPICE and (C) VCP. SPICE (20 ng) and VCP (200 ng) were incubated for 10 min with sheep erythrocytes (EAC142 cells). Guinea pig serum (EDTA treated) was then added for 30 min as a source of C3-C9. Percent inhibition of lysis was calculated in comparison to conditions without inhibitor. Representative experiment of three.

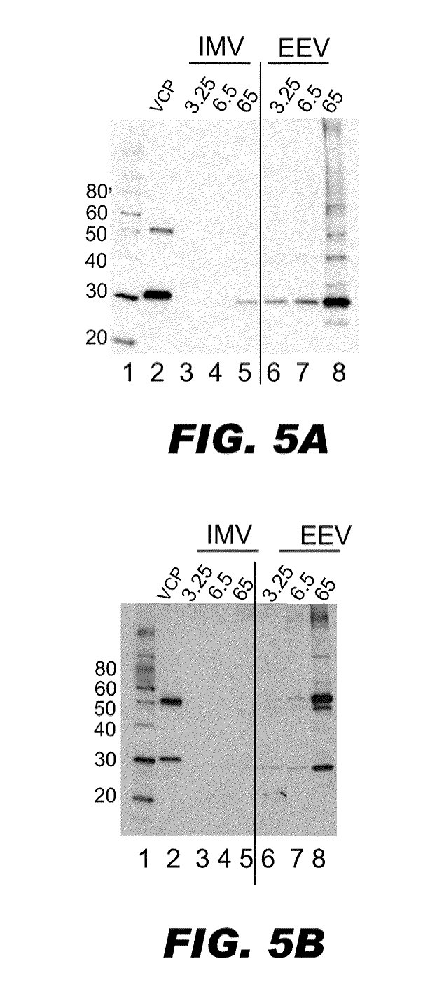

FIG. 5 depicts western blots showing that KL5.1 mAb detects PICE proteins deposited on infectious virions. Mammalian cells were infected with the vaccinia virus. Intracellular mature virus (IMV) and extracellular enveloped virus (EEV) particles were isolated and lysed. Following SDS-PAGE, the gel was probed (Western blot) with (A) polyclonal rabbit-anti-VCP Ab, or (B) KL 5.1 mAb. VCP standard in lane 2. As indicated, the number of plaque forming units (pfu) per lane applied was 3.25, 6.5, and 65×10⁴.

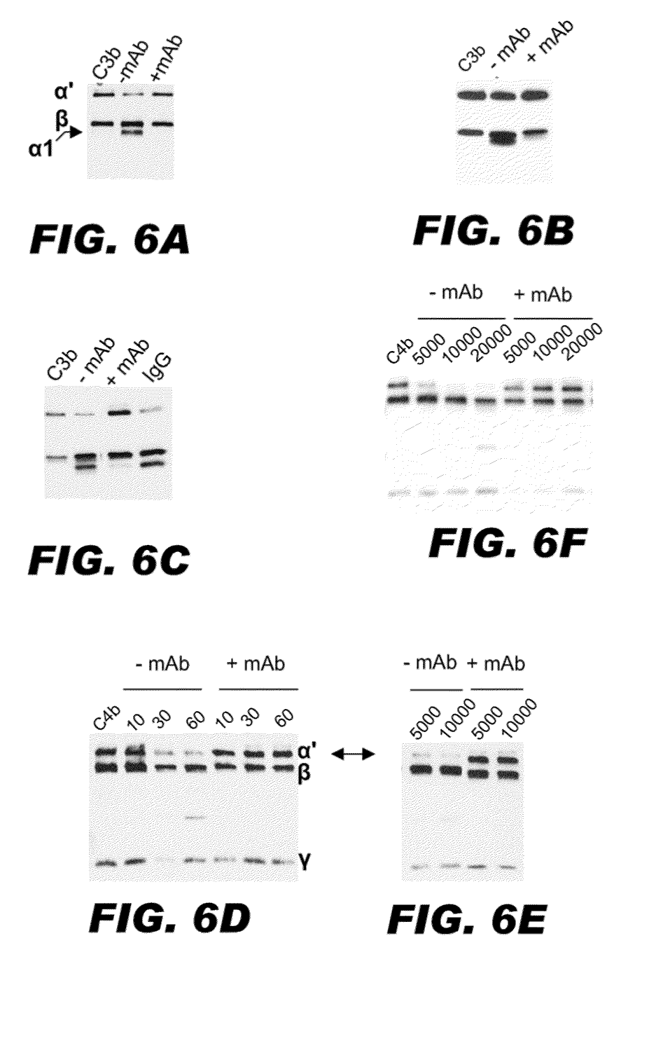

FIG. 6 depicts western blots showing that monoclonal Ab KL5.1 inhibits cofactor activity of SPICE, MOPICE and VCP. Biotinylated ligand (C3b or C4b) was incubated with factor I and the supernatants from CHO transfectants. Following electrophoresis on 10% SDS-PAG, Western blofting was performed using HRP-avidin. Loss of the α′ chain and development of α₁ cleavage fragments were monitored and are marked with an arrow. Longer exposures are required to visualize the α₂ fragments (not shown). (A) The mAb inhibits ability of SPICE to serve as a cofactor for C3b cleavage. The C3b control (no added cofactor) is shown in the first lane. (B) The mAb shows partial inhibiting of MOPICE and (C) VCP. mAb KL5.1 inhibits CA for C4b for (D) SPICE, (E) MOPICE, and (F) VCP.

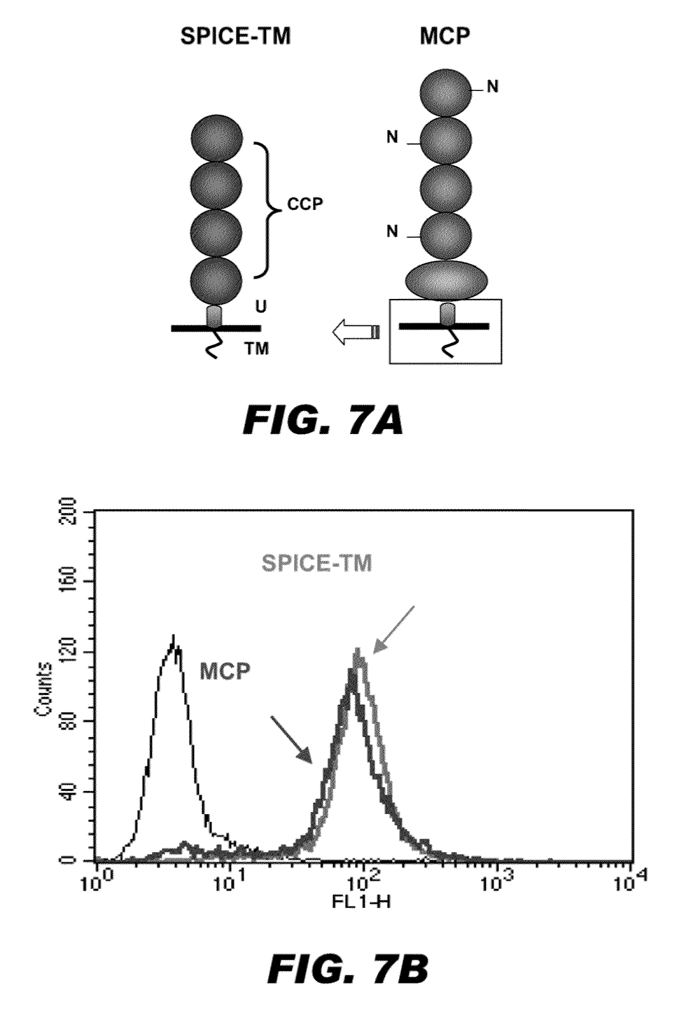

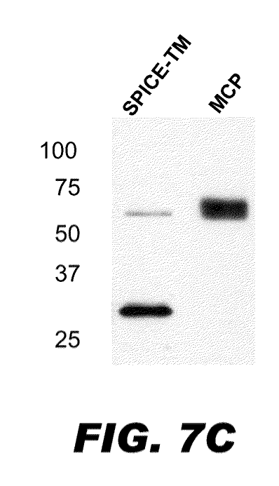

FIG. 7 depicts an illustration, a FACS plot, and a Western blot characterizing SPICE bearing the transmembrane domain of membrane cofactor protein (MCP; CD46). (A) Schematic diagram of SPICE-TM and MCP. Each is composed of four homologous modules called complement control protein repeats (CCPs). The human complement regulator, MCP, has three N-linked sugars (N). The CCP region of MCP is followed by an O-glycosylated domain (green oval), a 12 amino acid segment of unknown significance (U, purple), and a transmembrane domain with cytoplasmic tail (TM). The four CCPs of SPICE were fused to the U-segment, transmembrane domain and cytoplasmic tail of MCP. (B) These constructs were expressed in Chinese hamster ovary cells. Clones were selected for similar expression levels via flow cytometry. (C) Western blot showing the expected Mᵣ for each protein. Expression level was ˜25,000 copies per cell for both SPICE-TM and MCP as determined by flow cytometry and ELISA.

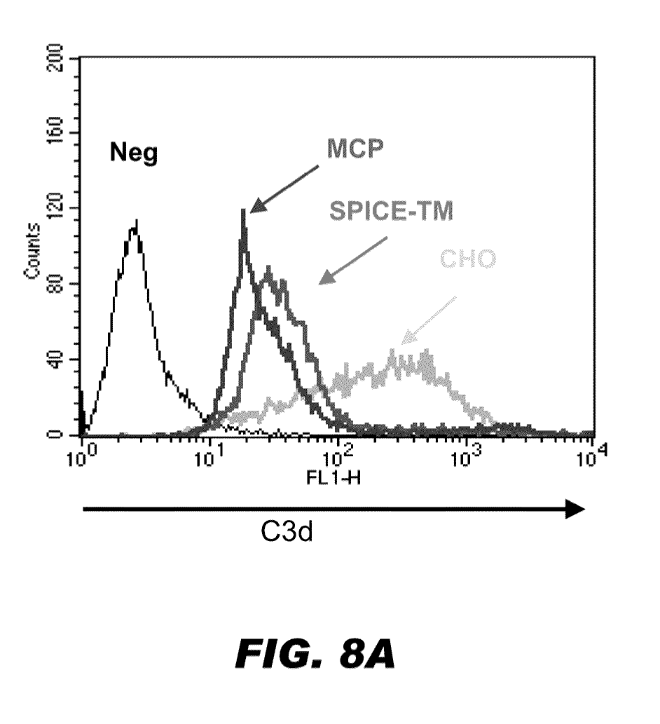

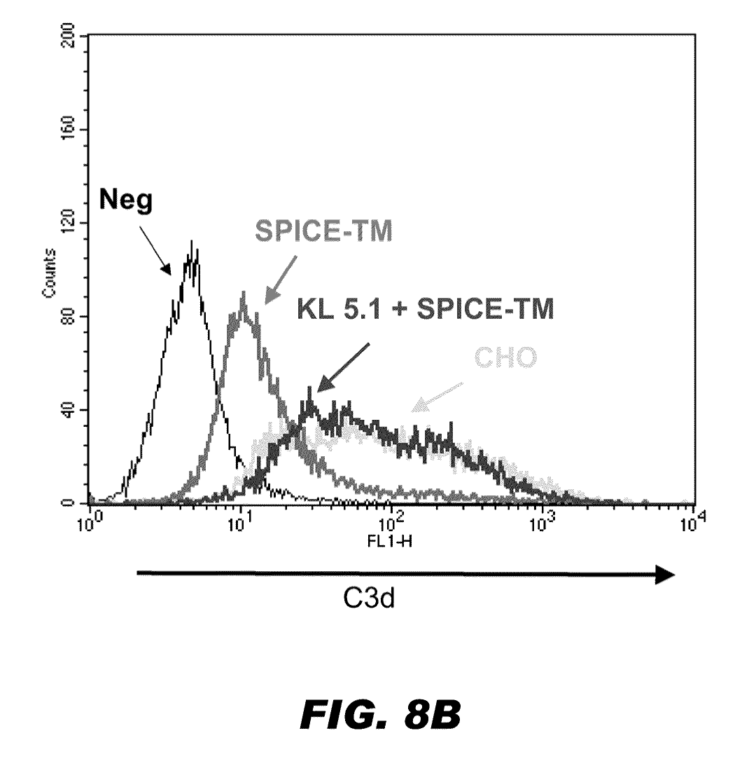

FIG. 8 depicts FACS plots showing an alternative pathway complement challenge of CHO cells expressing SPICE or MCP. (A) Transmembrane SPICE reduces C3 deposition similar to MCP after alternative pathway challenge. CHO control, transmembrane SPICE and transmembrane MCP expressing cells were sensitized with 0.5 mg/ml anti-CHO Ab followed by challenge with 10% C8-deficient serum for 45 min at 37° C. in GVB-MgEGTA. Deposition of C3 fragments was measured by FACS using a mAb to C3d followed by FITC rabbit anti-mouse IgG. Negative (Neg) control used an isotypic mAb. (B) Complement inhibitory activity of SPICE-TM is inhibited by mAb KL5.1. CHO cells, with or without transmembrane SPICE, were sensitized with 0.5 mg/ml anti-CHO Ab followed by incubation with 6.5 μg/ml of mAb KL5.1. Cells were subsequently complement “challenged” as described above. Negative (Neg) described above. Results shown are representative experiments from three to four performed.

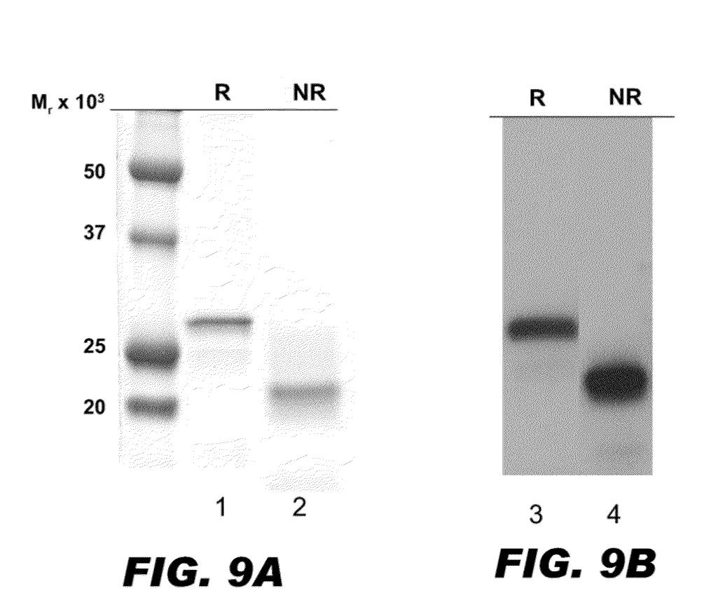

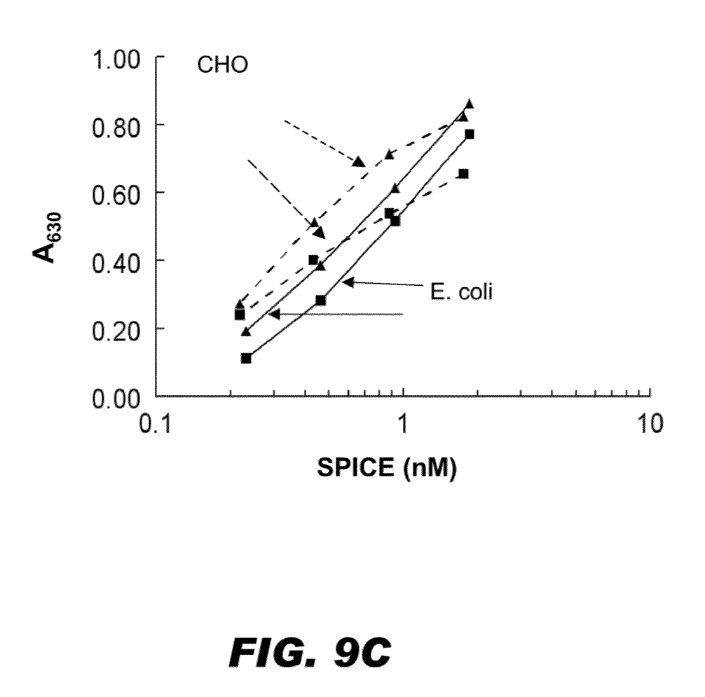

FIG. 9 depicts a Western blot and a graph characterizing SPICE produced recombinantly in an E. coli expression system. Electrophoresis on a 12% SDS-PAG (A, B). Reduced (lanes 1 and 3) and non-reduced (lanes 2 and 4) samples were analyzed by either (A) Coomassie blue staining (B) or by Western blotting with a polyclonal Ab. (C) To assess activity, SPICE binding to C3b/C4b was characterized in ELISA. A representative experiment from three is shown.

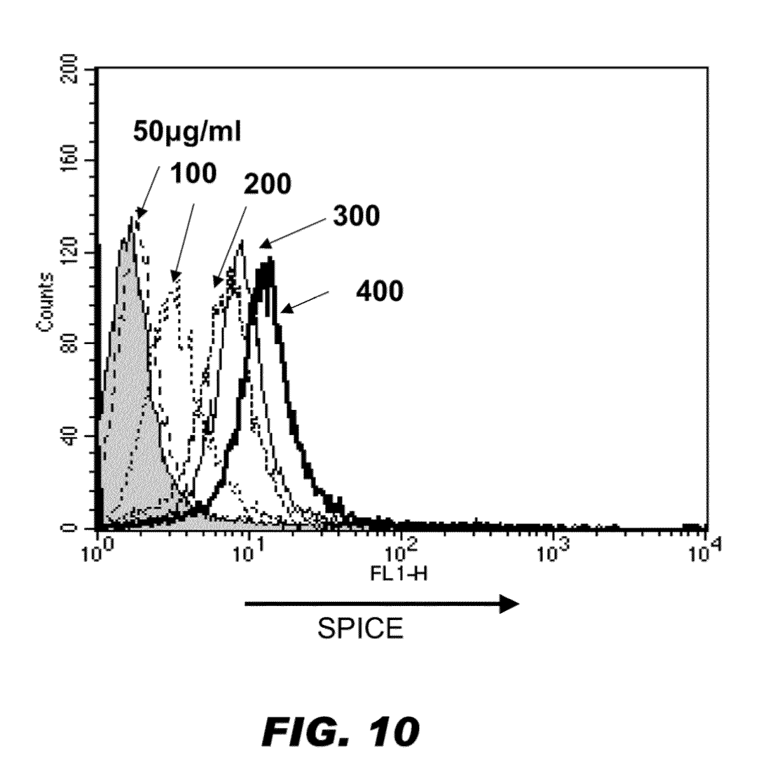

FIG. 10 depicts a FACS plot showing that recombinant SPICE produced in an E. coli expression system binds to CHO cells. To 5×10⁵ cells, purified SPICE was incubated at 50, 100, 200, 300 and 400 μg/ml in 50 μL at 37° C. for 30 min. SPICE binding was detected by FACS using a polyclonal Ab and a FITC-labeled secondary Ab. An isogenic IgG control is indicated by the shaded area. A representative experiment of three is shown.

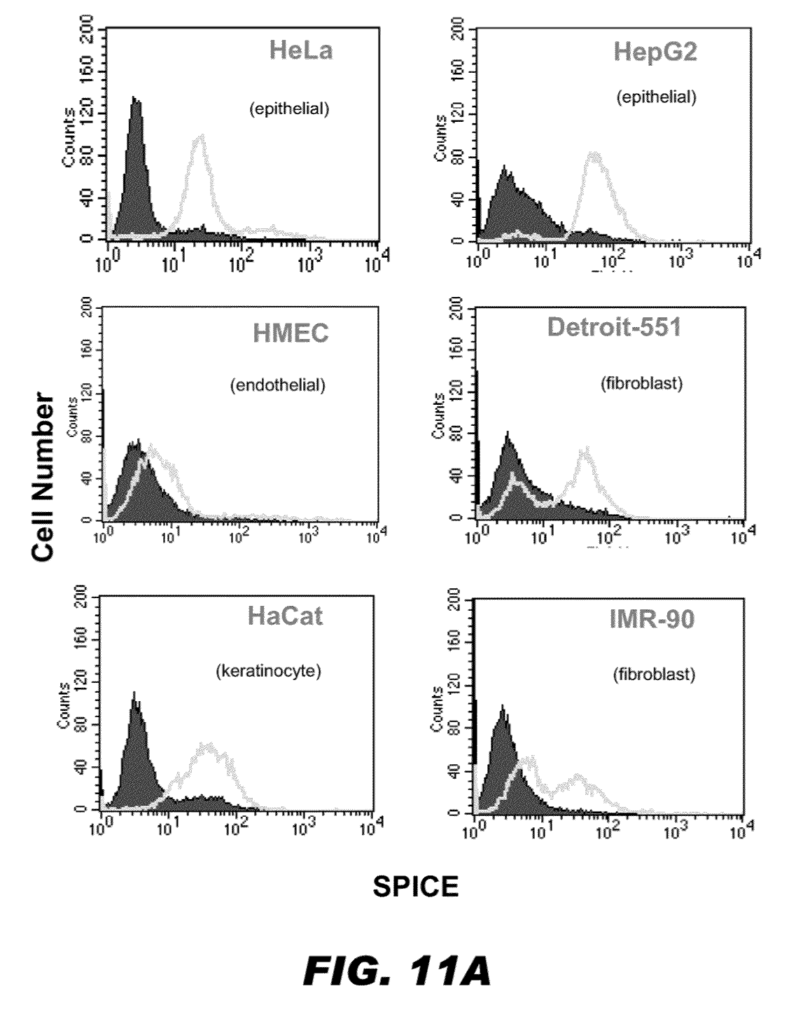

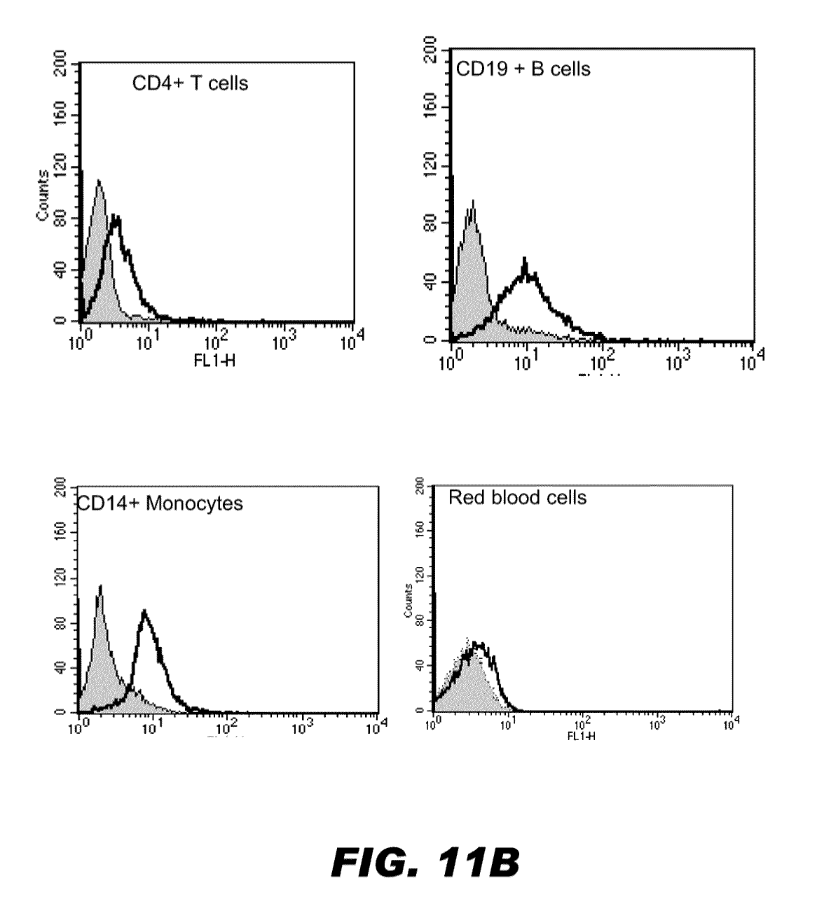

FIG. 11 depicts several different FACS plots demonstrating that SPICE attaches to multiple human cell types. (A) Cells were incubated with recombinant SPICE (200 μg/ml) for 1 h, harvested from flasks by treatment with EDTA (for adherent cells), washed, and assessed for SPICE binding using a rabbit polyclonal Ab and a FITC secondary Ab (light line). An IgG isotype control is shown (shaded). (B) CD4+ T cells, CD19+ B cells, CD14+ monocytes, and red blood cells were purified from peripheral blood and incubated with SPICE as in A. B cells and monocytes were pre-incubated with human IgG to inhibit Fc receptors and subsequently analyzed with a polyclonal Ab to SPICE. Detection was with a FITC-conjugated F(ab′)₂ secondary Ab for FACS analysis. SPICE bound to T cells was similarly detected, without preincubation with human IgG. Representative experiments from three to four are shown.

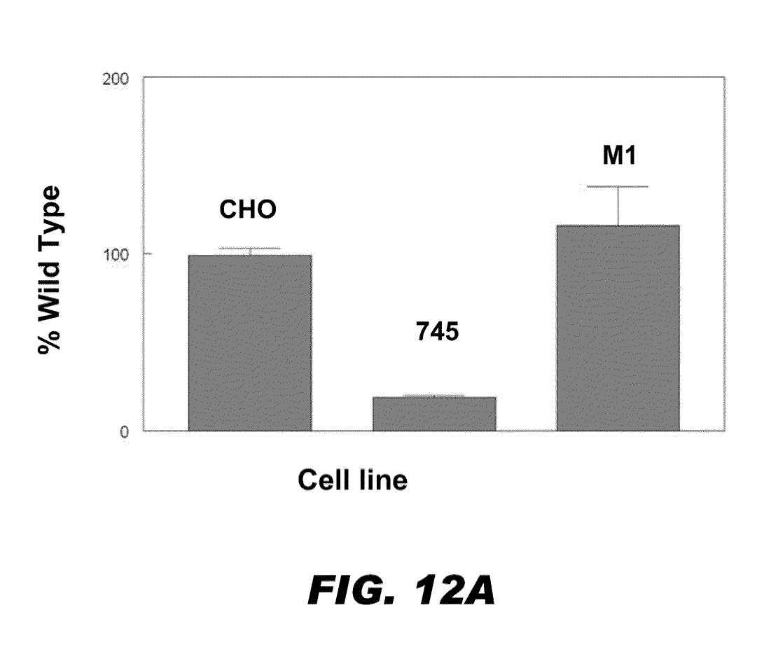

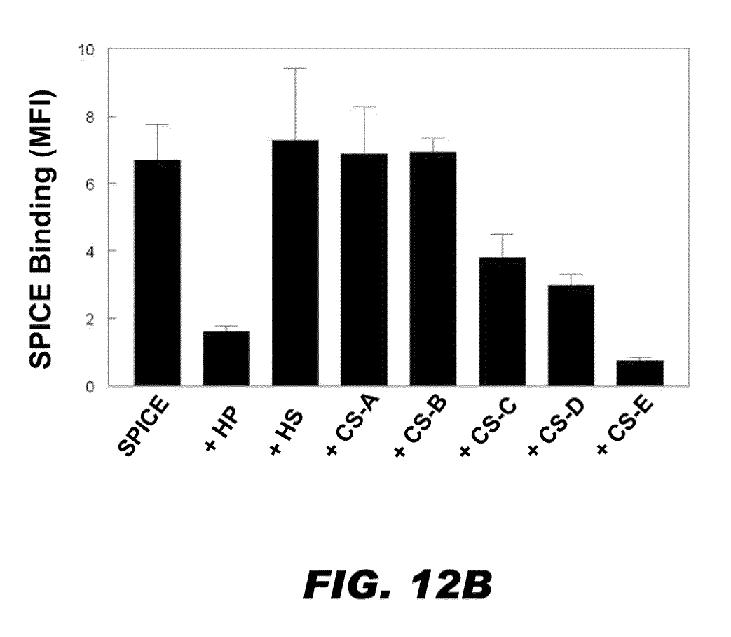

FIG. 12 depicts a graph showing that the binding of SPICE to cells is glycosaminoglycan (GAG)-dependent. (A) Soluble SPICE was incubated with wild-type (CHO) and mutant CHO cell lines defective for specific enzymes in GAG synthesis (745 or M1). Cells were detached with EDTA. Binding was detected by FACS analysis with a polyclonal Ab followed by incubation with a FITC-conjugated anti-rabbit IgG. (B) Heparin (HP) and chondroitin sulfate-E (CS-E) inhibit binding of SPICE to CHO cells. Soluble SPICE (200 μg/ml) was preincubated with soluble GAGs (5 μg/ml) followed by binding to CHO cells. The ability of soluble GAGs to inhibit SPICE binding was assessed by FACS with a polyclonal Ab followed by staining with a FITC-anti rabbit IgG. HP, heparin; HS, heparan sulfate, CS-A through CS-E are types of chondroitin sulfates. Data from (A) and (B) represent the mean+SEM for 3 and 5 experiments, respectively.

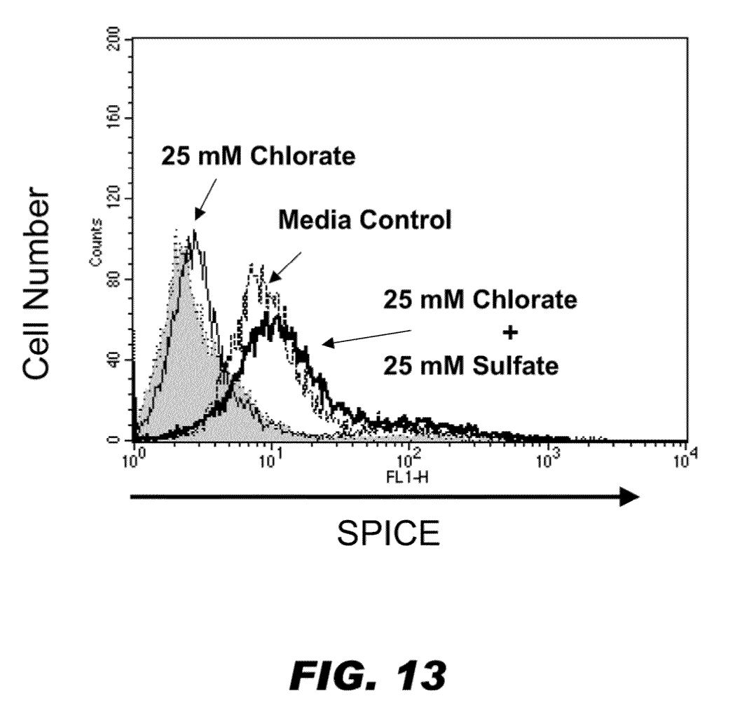

FIG. 13 depicts a FACS plot showing that GAG sulfation is critical for SPICE binding to CHO cells. CHO cells were incubated in sulfate-free Ham's F12 containing dialyzed FBS with 25 mM chlorate, 25 mM chlorate plus 25 mM sulfate, or normal Ham's F12 (media control). After harvesting cells by EDTA treatment, they were incubated with SPICE. SPICE binding was detected by FACS analysis using a polyclonal Ab and a FITC-labeled secondary Ab. Representative experiment from three is shown.

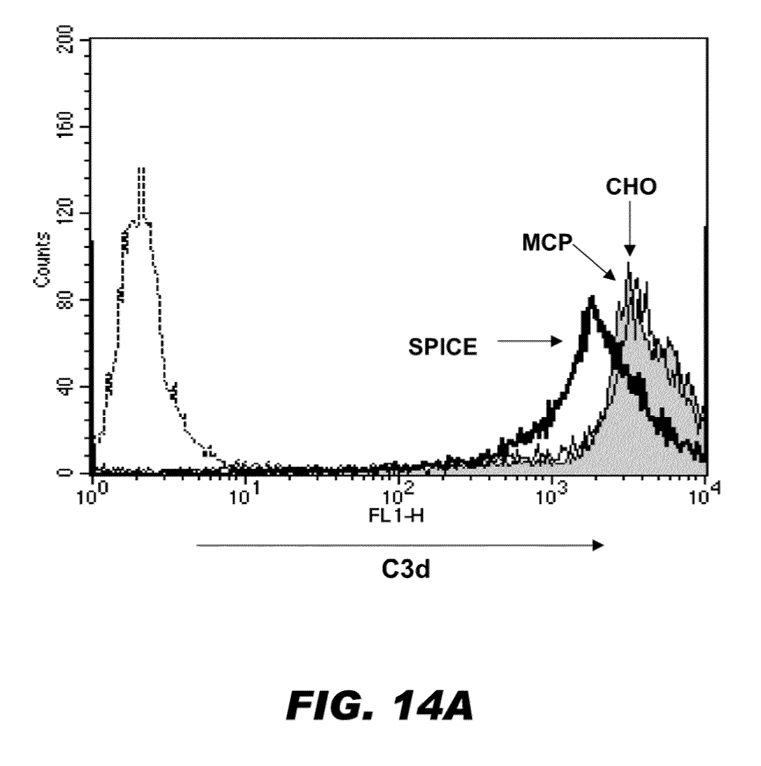

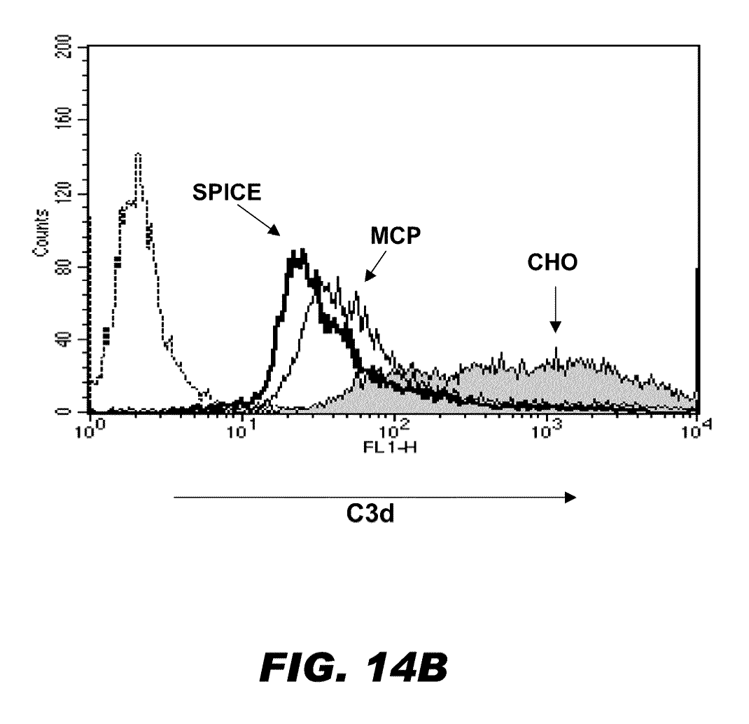

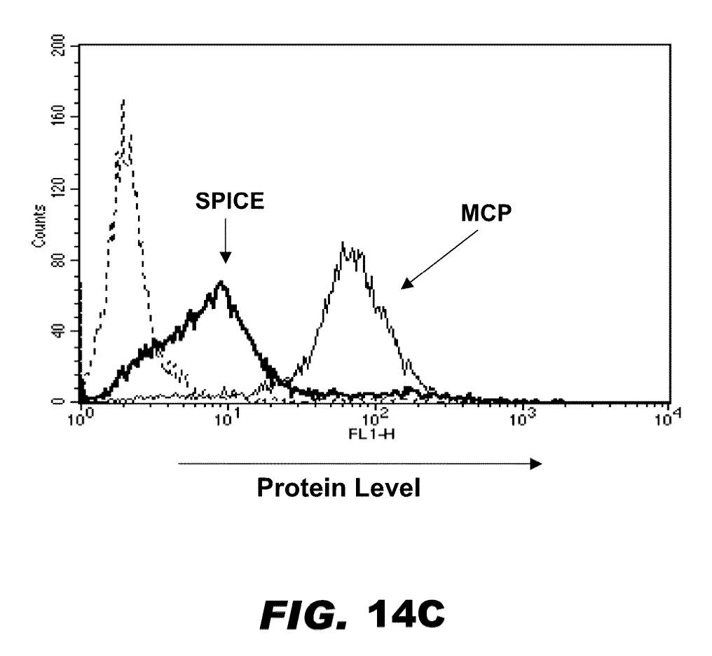

FIG. 14 depicts a series of FACS plots showing that SPICE reduces complement deposition following activation of the classical and alternative pathways. (A) C3 fragment deposition by classical pathway activation. CHO cells preincubated with SPICE (dark line) or without (shaded) and an MCP-expressing stable line (light line), were sensitized with 1 mg/ml anti-CHO Ab and subsequently challenged with 10% C8-deficient serum for 45 min at 37° C. in GVB⁺⁺. C3 fragment deposition was measured with a mAb to C3d. The dotted line to the left is a control in which there was no serum added. Mean fluorescent intensity (MFI) for C3b deposition for CHO control, SPICE, and MCP was 4351, 2106, and 4290, respectively. Shown is a representative experiment from three. (B) C3 fragment deposition by alternative pathway activation. Same designations as in (A). Cells were sensitized with 0.5 mg/ml anti-CHO Ab followed by incubation with 10% C8d serum for 45 min at 37° C. in GVB-MgEGTA buffer. MFI are: CHO, 630; SPICE, 28; MCP, 39. (C) The quantity of SPICE and MCP present on CHO cells was monitored using a rabbit polyclonal Ab and mAbs (not shown) and each demonstrated ˜10 fold more MCP than SPICE-GAG. Shown for (A), (B), and (C) are representative experiments from three to four conducted.

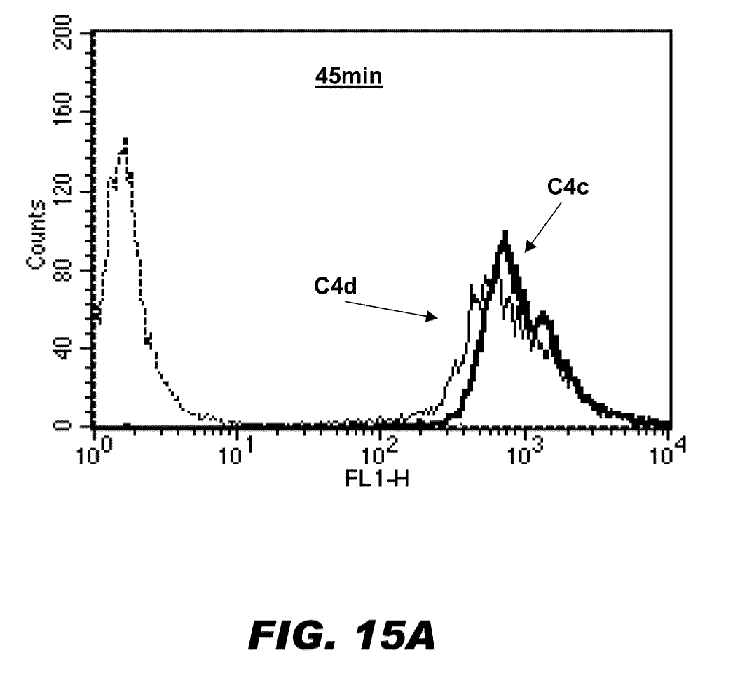

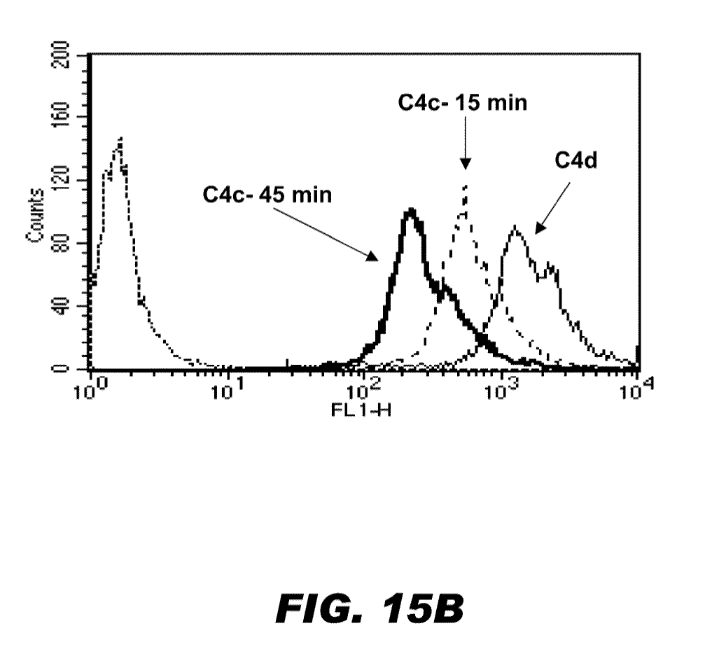

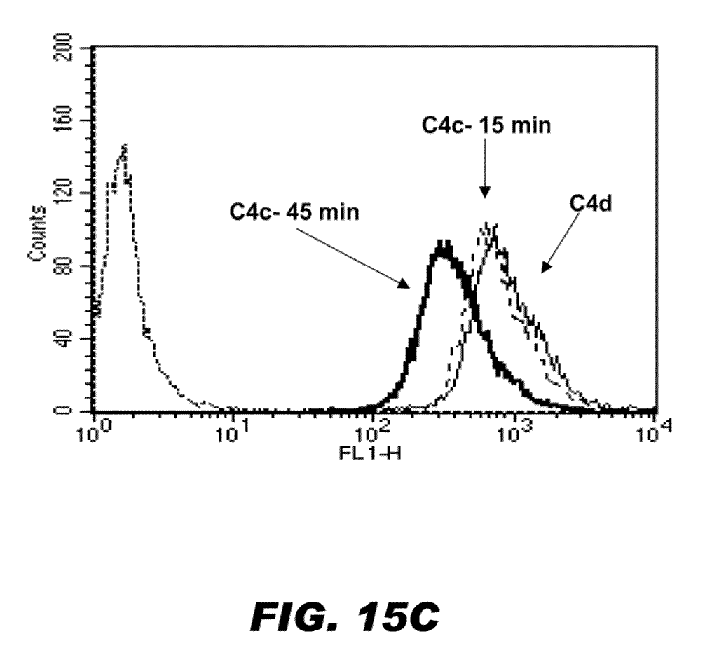

FIG. 15 depicts a series of FACS plots showing that SPICE cleaves C4b deposited on complement challenged CHO cells. Comparison of C4b cleavage by SPICE versus an MCP-expressing CHO clone. C4b cleavage was analyzed via FACS using a mAb to C4c (heavy and dashed lines) and C4d (light line). To activate complement, cells were sensitized with 1 mg/ml of anti-CHO Ab followed by incubation with 10% C8-deficient serum for 15 or 45 min in GVB⁺⁺ buffer. The dotted line on the left in the histograms indicates a condition without serum exposure. (A) CHO cells without an inhibitor show no cleavage of C4b after 45 min. (B) CHO cells with deposited SPICE show dose-dependent loss of C4c fragment. (C) MCP-expressing CHO clone also shows dose-dependent loss of C4c fragment, but to a lesser extent than SPICE. A representative experiment of three is shown.

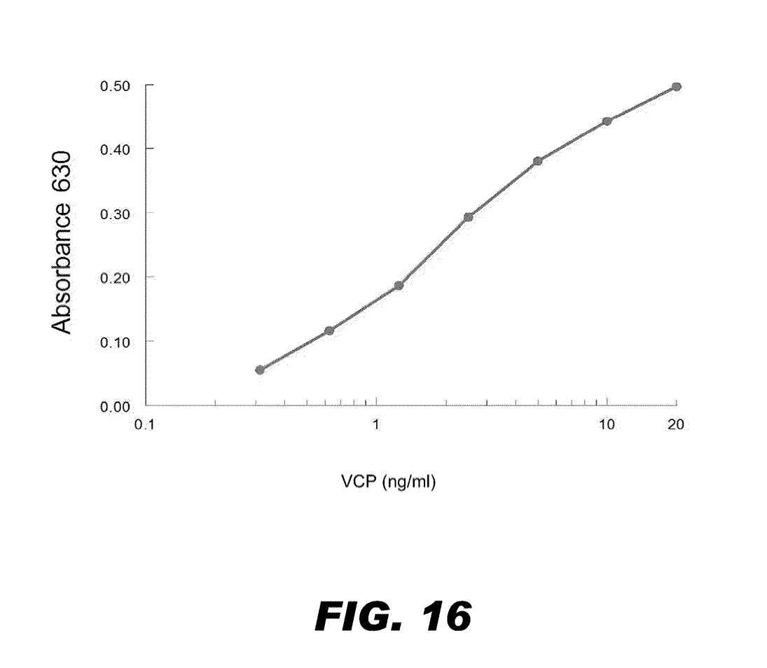

FIG. 16 depicts a graph showing mAb KL5.1 as a capture antibody in an ELISA to quantify PICES. The mAb was adsorbed to microtiter wells and dilutions of VCP added followed by detection with rabbit polyclonal anti-VCP antibody (Ab) and HRP anti-globulin Ab. Negative control (background) was subtracted. Lower limits of detection were 0.5 to 1.0 ng/ml.

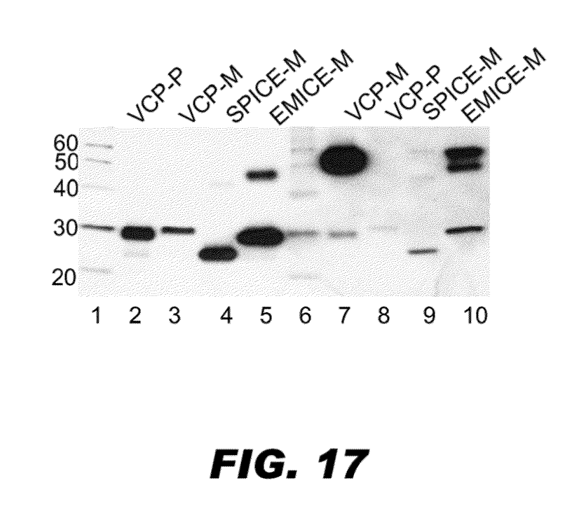

FIG. 17 depicts a Western blot showing detection of PICES with KL5.1. PICE samples were electrophoresed on 12% SDS-PAG, transferred to nitrocellulose, and probed with either the rabbit anti-VCP Ab (lanes 1-5) or KL5.1 mAb (lanes 6-10). Lanes 1 and 6 are the mol wt standards in kDa. Abbreviations: —P, indicates PICE protein produced in Pichia, -M, produced in mammalian (Chinese hamster ovary cells) expression system, EMICE, ectromelia (mousepox) inhibitor of complement enzymes.

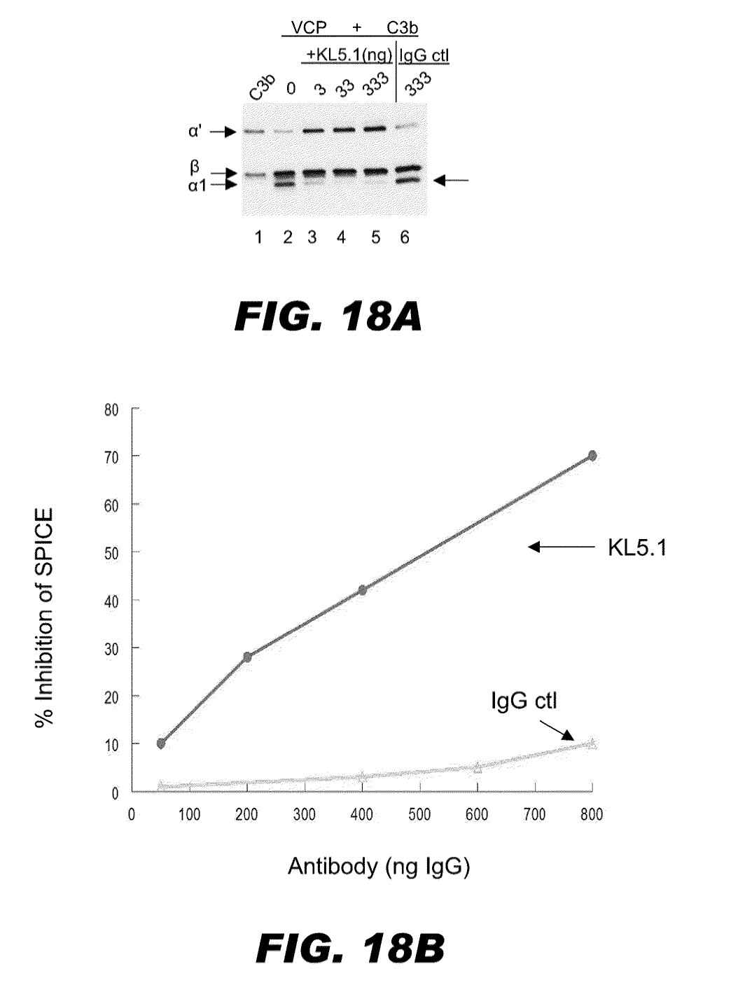

FIG. 18 depicts a Western blot and a graph showing that mAb KL5.1 inhibits PICE function. (A) The mAb KL5.1 inhibits C3b cofactor activity. Control lane 1, C3b. Lanes 5-8 contain C3b+VCP (and factor I) with or without mAb KL5.1 (ng/reaction). The expected cleavage fragments indicated by an arrow demonstrates a dose-dependent decrease with mAb. IgG ctl, mAb to human complement regulatory protein (CD46) with the same isotype (IgG1) shows IgG does not interfere with cleavage of VCP. (B) The mAb KL5.1 inhibits decay accelerating activity (DAA). Sheep erythrocytes were coated with human complement and inhibition of lysis was measured. SPICE (20 ng) was added to each condition. SPICE activity was set at 100%.

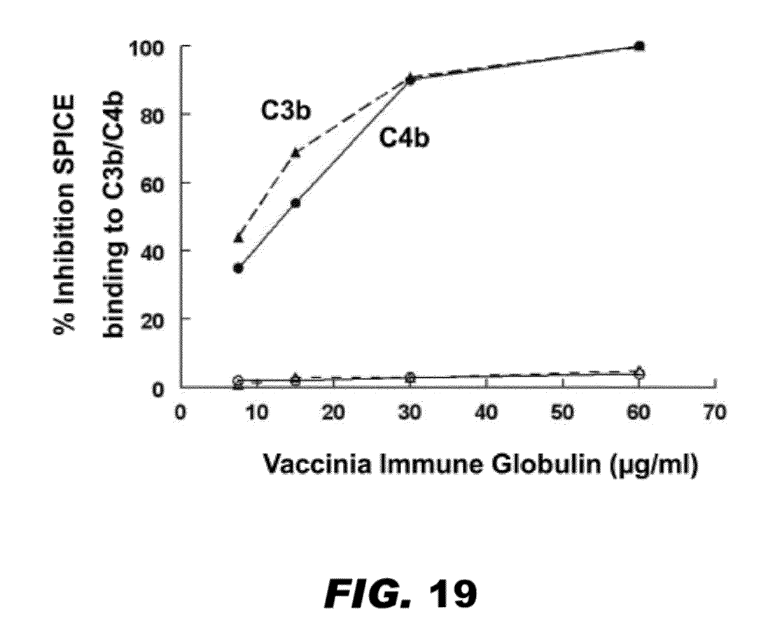

FIG. 19 depicts a graph showing the presence of function-blocking antibodies to SPICE in Vaccinia Immune Globulin (VIG). SPICE supernatant (10 ng/ml) expressed by CHO cells was pre-incubated with varying concentrations of VIG (filled triangles and circles) or control IgG (open triangles or circles) for 30 min at 37° C. then incubated on wells coated with C3b (triangles) or C4b (circles) for 1 hr 37° C. Detection of SPICE binding was by rabbit Ab and HRP anti-globulin. Percent inhibition obtained by comparing binding to non-IgG control. Representative experiment of three.

### DETAILED DESCRIPTION OF THE INVENTION

The present invention provides antibodies that bind to and substantially inhibit the activity of at least one poxvirus complement inhibitor. The invention also provides methods of use for the antibodies. For instance, the antibodies may be used in methods for detecting a poxvirus complement inhibitor and methods for decreasing the activity of a poxvirus complement inhibitor.

### I. Antibodies

An antibody of the invention binds to and substantially inhibits the activity of at least one poxvirus complement inhibitor. As used herein, the term “antibody” may refer to a monoclonal antibody, a polyclonal antibody, a recombinant antibody, a single chain antibody, or an antibody fragment. Suitable monoclonal antibodies may include chimeric antibodies and humanized antibodies. Suitable recombinant antibodies may include chimeric antibodies, humanized antibodies, recombinant antibody fragments, i.e. Fv fragments, single chain Fv fragments, disulfide stabilized Fv (dsFv) fragments, single domain antigen binding fragments, i.e. VHs, recombinant llama heavy chain antibody fragments, camelized antibodies, and antibody fusion proteins. Non-limiting examples of suitable single chain antibodies may include single chain Fv fragments and single domain antigen binding fragments, i.e. VHs. Suitable antibody fragments may include, but are not limited to, single chain antibody fragments, i.e., single chain Fv fragments, recombinant llama heavy chain antibody fragments, recombinant antibody fragments, i.e. Fv fragments, disulfide stabilized Fv (dsFv) fragments, single domain antigen binding fragments, i.e., VHs, Fc fragments, and Fab fragments. In one embodiment, an antibody of the invention is a monoclonal antibody.

Antibodies of the invention may be made in accordance with methods generally known in the art as detailed below and in the examples.

### (a) Poxvirus Complement Inhibitor

As stated above, an antibody of the invention binds to a poxvirus complement inhibitor. Generally speaking, a poxvirus complement inhibitor is a viral protein capable of substantially inhibiting the host complement cascade. A poxvirus complement inhibitor may inhibit complement at several different points in the cascade. Usually, though, a poxvirus inhibitor will substantially inhibit the host C3 convertase or the C5 convertase. The C3 and C5 convertases may derive from either the classical, lectin, or alternative pathways. Stated another way, a poxvirus complement inhibitor will typically act on the complement cascade so as to inhibit the cleavage of C3 to form C3b, the cleavage of C4 to form C4b, or the cleavage of C5 to form C5b.

Poxviruses that encode a poxvirus complement inhibitor may be from the subfamily Chordopoxvirinae. For instance, a poxvirus complement inhibitor may be encoded by a virus from the genus Orthopoxvirus, including the species buffalopox, camelpox, cowpox, ectromelia, monkeypox, rabbitpox, raccoonpox, sealpox, skunkpox, vaccinia, variola, and volepox. Non-limiting examples of poxvirus complement inhibitors may include the smallpox inhibitor of complement enzymes (SPICE), the monkeypox inhibitor of complement enzymes (MOPICE), the vaccinia complement control protein (VCP), or the ectromelia inhibitor of complement enzymes (EMICE). In one embodiment, an antibody of the invention may bind to and substantially inhibit SPICE. In another embodiment, an antibody of the invention may bind to and substantially inhibit MOPICE. In yet another embodiment, an antibody of the invention may bind to and substantially inhibit VCP. In still yet another embodiment, an antibody of the invention may bind to and substantially inhibit EMICE.

In certain embodiments, an antibody of the invention may bind to and substantially inhibit at least two, at least three, or at least four poxviral complement inhibitors. For instance, in some embodiments, an antibody of the invention may bind to and substantially inhibit at least two, at least three, or at least four poxviral complement inhibitors selected from the group comprising SPICE, MOPICE, VCP or EMICE.

### (b) Inhibiting the Activity of a Poxvirus Complement Inhibitor

An antibody of the invention substantially inhibits the activity of a poxvirus complement inhibitor. In some embodiments, an antibody of the invention may inhibit the cofactor activity of a poxvirus complement inhibitor. The phrase “cofactor activity,” as used herein, refers to the limited proteolytic degradation of C3b and C4b that results from the combined activity of the poxvirus complement inhibitor and a serine proteinase, such as factor I. In other embodiments, an antibody of the invention may substantially inhibit the decay accelerating activity (DAA) of a poxvirus complement inhibitor. The phrase “decay accelerating activity,” as used herein, refers to the dissociation, mediated by a poxvirus complement inhibitor, of the catalytic serine protease domain from complement-activating enzyme complexes or convertases. In certain embodiments, an Ab of the invention may inhibit both the CA and DAA of a poxviral complement inhibitor. The term “substantially inhibiting,” as used herein, refers to inhibiting the CA or DAA of a poxvirus complement inhibitor about 10, 20, 30, 40, 50, 60, 70, 80, 90, or 100% compared to the same complement inhibitor not in the presence of an antibody of the invention. Methods of measuring CA and DAA are known in the art. For instance, see the Examples.

### (c) Detectable Marker

In certain embodiments, an antibody of the invention may be labeled with a detectable marker. The marker may be either non-covalently or covalently joined to an antibody of the present invention by methods generally known in the art. Detectable markers suitable for use in the invention generally comprise a reporter molecule or enzyme that is capable of generating a measurable signal. By way of non-limiting example, such detectable markers may include a chemiluminescent moiety, an enzymatic moiety (e.g. horse-radish peroxidase), a fluorescent moiety (e.g. FITC) or a radioactive moiety. Additionally, in some embodiments, an antibody of the invention may be labeled with avidin or biotin.

### (d) Production of an Antibody

An antibody of the invention may be generated using a poxvirus complement inhibitor, or a fragment thereof, as an immunogen using methods that are well known in the art. Generally speaking, if a fragment of a poxvirus complement inhibitor were to be used as an immunogen, the fragment should comprise a binding site for either C3b or C4b, or a site known or suspected to be involved in CA or DAA. Identification and selection of an antibody that can inhibit activity may be performed using methods commonly known in the art. For more details, see the Materials and Methods for Examples 1-5.

For the production of polyclonal antibodies, various hosts including goats, rabbits, rats, mice, humans, and others may be immunized by injection with a poxvirus complement inhibitor or peptide thereof, as detailed above, that has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and corynebacterium parvum are especially preferable.

Monoclonal antibodies that bind to and substantially inhibit the activity of a poxvirus complement inhibitor may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity may be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-45). Alternatively, techniques described for the production of single chain antibodies may be adapted, using methods known in the art, to produce single chain antibodies that bind to and substantially inhibit the activity of a poxvirus complement inhibitor. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments that contain specific binding sites for a poxvirus complement inhibitor or fragments thereof may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

In the production of antibodies, screening for the desired antibody may be accomplished by techniques known in the art, e.g. ELISA (enzyme-linked immunosorbent assay). Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a complement enzyme (e.g. C3b) and its specific antibody.

Various methods such as Scatchard analysis in conjunction with radioimmunoassay techniques may be used to assess the affinity of antibodies for a poxvirus complement inhibitor. Affinity is expressed as an association constant, Kₐ, which is defined as the molar concentration of anti-poxvirus complement inhibitor complex divided by the molar concentrations of free antigen and free antibody under equilibrium conditions. The Kₐ determined for a preparation of polyclonal antibodies, which are heterogeneous in their affinities for multiple epitopes, represents the average affinity, or avidity, of the antibodies for a poxvirus complement inhibitor. The Kₐ determined for a preparation of monoclonal antibodies, which are monospecific for a particular epitope, represents a true measure of affinity. High-affinity antibody preparations with Kₐ ranging from about 10⁹ to 10¹² L/mole are preferred for use in immunoassays in which the poxvirus complement inhibitor-antibody complex must withstand rigorous manipulations. Low-affinity antibody preparations with Kₐ ranging from about 10⁶ to 10⁷ L/mole are preferred for use in immunopurification and similar procedures that ultimately require dissociation of the poxvirus complement inhibitor, preferably in active form, from the antibody.

The titer and avidity of polyclonal antibody preparations may be further evaluated to determine the quality and suitability of such preparations for certain downstream applications. Procedures for evaluating antibody specificity, titer, and avidity, and guidelines for antibody quality and usage in various applications, are generally available.

### (e) Pharmaceutical Compositions

An antibody of the invention may be incorporated into a pharmaceutical composition suitable for administration to a subject. Typically, the pharmaceutical composition comprises an antibody or antibody fragment of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The pharmaceutical compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Pharmaceutical compositions may be sterile and are typically stable under the conditions of manufacture and storage. The composition may be formulated as a solution, microemulsion, dispersion, liposome, or other ordered structure suitable to high drug concentration. Sterile injectable solutions can be prepared by incorporating the active compound (i.e., antibody or antibody fragment) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those detailed above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution may be maintained, for example, by the use of a coating such as lecithin, by the maintenance of the required particle size in the case of dispersion and by the use of surfactants. Prolonged absorption of injectable compositions may be achieved by including an agent that delays absorption, for example, monostearate salts and gelatin, in the composition.

An antibody of the invention, or a pharmaceutical composition comprising an antibody of the invention, may be administered to a subject. An antibody of the present invention may be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody of the invention may be orally administered, for example, with an inert diluent or an assimilable edible carrier. The composition (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

A pharmaceutical composition may also comprise one or more additional anti-poxvirus antibodies. Alternatively, an antibody of the invention may be coadministered with one or more anti-poxvirus antibodies.

### (f) Antibody Conjugates

In some embodiments of the invention, an antibody may be conjugated to a complex, such as a therapeutic complex or an imagining complex. Methods of conjugating antibodies to various complexes are known in the art.

### II. Methods

Another aspect of the invention encompasses methods for detecting a poxvirus complement inhibitor and decreasing the activity of a poxvirus complement inhibitor. Each is discussed in more detail below.

### (a) Detecting a Poxvirus Complement Inhibitor

In one embodiment, the invention encompasses a method for detecting a poxvirus complement inhibitor in a sample. Such a method generally comprises contacting the sample with an antibody of the invention and detecting association of the antibody with a poxvirus complement inhibitor. In some embodiments, the method further comprises quantifying a poxvirus complement inhibitor in a sample. In other embodiments, the method further comprises purifying a poxvirus complement inhibitor from a sample. A suitable sample may be any sample that might comprise a poxvirus complement inhibitor. For instance, a sample may be a bodily fluid, a bodily tissue, a cell culture, or a cell culture supernatant. In one embodiment, the sample is a bodily fluid. For instance, the sample may be a blood sample. A blood sample may be a whole blood sample, a plasma sample, or a serum sample.

Methods of detecting association of the antibody with a poxvirus complement inhibitor are commonly known in the art. For more details, see the Examples. For instance, an immunoassay may be used to detect and/or quantify a poxvirus complement inhibitor in a sample with an antibody of the invention. The immunoassays that may be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

In one embodiment, the invention provides a method for detecting a poxvirus complement inhibitor using an ELISA. Methods of performing ELISAs are known in the art, and are detailed in the Examples. An antibody of the invention may be used as a capture antibody in an ELISA. Alternatively, an antibody of the invention may be used as a primary antibody in an ELISA.

In another embodiment, the invention provides a method for detecting a poxvirus complement inhibitor via a Western blot. Methods of performing Western blots are known in the art, and are detailed in the Examples.

In yet another embodiment, the invention provides a method for detecting a poxvirus complement inhibitor by immunoprecipitating a poxvirus complement inhibitor from a sample with an antibody of the invention. In a further embodiment, the poxvirus complement inhibitor may be purified from a sample by immunoprecipitation. For instance, an affinity matrix may be provided that comprises an antibody of the invention that selectively binds to poxvirus complement inhibitor. Typically, a biological sample is contacted with the affinity matrix to produce an affinity matrix-poxvirus complement inhibitor complex. The affinity matrix-poxvirus complement inhibitor complex is separated from the remainder of the biological sample and the poxvirus complement inhibitor is released from the affinity matrix to form a purified poxvirus complement inhibitor. Suitable affinity matrixes are known in the art and may include a solid support, a bead, or a resin.

The invention also provides a method of detecting a dimeric form of a poxvirus complement inhibitor in a sample. Such a method generally comprises contacting the sample with an antibody of the invention and detecting association of the antibody with a dimeric poxvirus complement inhibitor. Moreover, the invention provides a method of detecting a poxvirus complement inhibitor on the surface of infectious virions. For instance, an antibody of the invention may be used to detect a poxvirus complement inhibitor on the surface of an extracellular enveloped virion or an intracellular immature virion. Such a method generally comprises contacting the infectious virion, or a sample comprising an infectious virion, with an antibody of the invention and detecting association of the antibody with a poxvirus complement inhibitor.

The invention also encompasses diagnostic methods. For example, the invention provides a method of detecting whether a subject is infected with a poxvirus. The method comprises contacting a sample from the subject with an antibody of the invention and detecting association between the antibody and a poxvirus complement inhibitor. Similarly, the invention encompasses a method of monitoring the success of vaccination against a poxvirus infection in a subject. The method generally comprises detecting and quantifying a poxvirus complement inhibitor in a sample from a subject, where the quantity of the complement inhibitor is indicative of the success of the vaccination.

In general, methods of the invention may be used for any subject capable of being infected with a poxvirus. For instance, the subject may be human, but may also be a non-human primate, a companion animal such as a dog or cat, a livestock animal such as a cow, horse, sheep or pig, or a rodent, such as a mouse, rat or guinea pig.

The method of each of the above embodiments may be performed under reducing or non-reducing conditions.

### (b) Decreasing the Activity of a Poxvirus Complement Inhibitor

In another embodiment, the invention encompasses a method for decreasing the activity of a poxvirus complement inhibitor. Generally speaking, the method comprises contacting a poxvirus complement inhibitor with an antibody of the invention. In one embodiment, the invention encompasses a method for decreasing the CA of a poxvirus complement inhibitor. In another embodiment, the invention encompasses a method for decreasing the DAA of a poxvirus complement inhibitor. In yet another embodiment, the invention encompasses a method for decreasing both the CA and the DAA of a poxvirus complement inhibitor. The term “decreasing,” as used herein, refers to decreasing the CA or DAA of a poxvirus complement inhibitor about 10, 20, 30, 40, 50, 60, 70, 80, 90, or 100% compared to the same complement inhibitor not in the presence of an antibody of the invention.

In certain embodiments, the activity of a poxvirus complement inhibitor may be decreased in vivo. For instance, an antibody of the invention, or a pharmaceutical composition comprising an antibody of the invention may be administered to a subject as detailed in section I above. Generally speaking, the dose of antibody administered depends in part on the subject, the reason for the administration, and the pharmaceutical composition. Dosage regimens may be adjusted to provide the optimum desired response. For example, a single bolus may be administered, several divided doses may be administered over time, or the dose may be proportionally reduced or increased as indicated by the exigencies of the situation.

An exemplary, non-limiting range for an effective amount of an antibody of the invention may be 0.1-20 mg/kg, more preferably 1-10 mg/kg. It is to be noted that dosage values may vary with the type and severity of the situation. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the invention encompasses a method for treating a poxviral infection in a subject. In another embodiment, the invention encompasses a method for treating an adverse reaction to a poxvirus vaccination in a subject. The above methods typically comprise administering an antibody of the invention to the subject to decrease the activity of a poxviral complement inhibitor. In this context, treating may refer to decreasing the viral load of a subject, or alleviating one or more symptoms of infection. In some embodiments of these methods, an antibody of the invention may be administered with one or more other anti-poxvirus antibodies.

Suitable subject may be those detailed in section II(a) above. In particular, suitable subjects may include those at risk for the development of a poxvirus infection, subjects exposed to a poxvirus, and subjects infected with a poxvirus.

### (c) Kits Comprising an Antibody of the Invention

A further aspect of the invention is a kit comprising an antibody of the invention for use in vitro or in vivo for detecting a poxvirus complement inhibitor or decreasing the activity of a poxvirus complement inhibitor. The components of the kits may be packaged either in aqueous medium or in lyophilized form. When the antibodies (or fragments thereof) are used in the kits in the form of conjugates in which a label or a therapeutic moiety is attached, such as a radioactive metal ion or a therapeutic drug moiety, the components of such conjugates may be supplied either in fully conjugated form, in the form of intermediates or as separate moieties to be conjugated by the user of the kit. The kits will also typically include instructions, the contents of which will vary depending upon the use of the kit.

### Definitions

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” in some embodiments denotes that a protein gives rise to essentially one band in an electrophoretic gel. Preferably, it means that the protein is at least 85% pure, more preferably at least 95% pure, and most preferably at least 99% pure. “Purify” or “purification” in other embodiments means removing at least one contaminant from the composition to be purified. In this sense, purification does not require that the purified compound be 100% pure.

The term “sample” or “biological sample” is used in its broadest sense. Depending upon the embodiment of the invention, for example, a sample may comprise a bodily fluid; an extract from a cell, chromosome, organelle, or membrane isolated from a cell; a cell; genomic DNA, RNA, or cDNA, in solution or bound to a substrate; a tissue; a tissue print or any other material isolated in whole or in part from a living subject. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Materials and Methods for Examples 1-5

Molecular engineering and expression of SPICE, VCP, chimeras and mutants. As previously described, the gene for VCP was PCR amplified using genomic DNA from the vaccinia Western Reserve and cloned into the plasmid pSG5 (Stratagene) (Liszewski, M. K. et al (2006) J. Immunol 176:3725). SPICE was constructed from VCP by mutation of the 11 amino acids using nine primers (Integrated DNA Technologies) and the QuikChange™ Multi Site-Directed Mutagenesis kit. During this process, eight SPICE-VCP chimeras were generated and subsequently employed in our studies.

Point mutations in SPICE were produced utilizing the QuikChange™ Site-Directed Mutagenesis kit. The fidelity of all clones was verified by DNA sequencing. Proteins were expressed transiently in Chinese hamster ovary (CHO) cells using Fugene-6 (Roche Molecular Biochemicals).

ELISA. The quantity of poxviral proteins was determined by ELISA as described (Liszewski, M. K. et al (2006) J. Immunol 176:3725). Briefly, the capture mAb, 5A10 (gift of Ariella Rosengard) (15, Liszewski, M. K. et al (2006) J. Immunol 176:3725) or mAb KL5.1 (see below) was coated at 5 μg/ml overnight at 4° C. and then inhibited for 1 h at 37° C. (1% BSA and 0.1% Tween 20 in PBS). Samples and standards (VCP) were incubated for 1 h at 37° C. and then washed with PBS containing 0.05% Tween 20. Next, a rabbit anti-VCP antiserum that cross-reacts with SPICE was applied for 1 h at 37° C. After washing, HRP-coupled donkey anti-rabbit IgG (Jackson ImmunoResearch Laboratories) was added and incubated for 1 h at 37° C. After washing, TMB substrate (Pierce Biotechnology) was added and absorbance (630 nm) measured in an ELISA reader.

Functional Assessment. Ligand binding, cofactor, and decay accelerating assays have been described (Liszewski, M. K. et al (2006) J. Immunol 176:3725). Briefly, for C3b and C4b binding, an ELISA format was used in which human C3b or C4b was coated overnight on wells and then inhibited (Liszewski, M. K. et al (2006) J. Immunol 176:3725). Samples and recombinant VCP (as standard) were incubated 1 h 37° C. and then washed. Next, rabbit anti-VCP antiserum was applied for 1 h at 37° C., and HRP-coupled donkey anti-rabbit IgG (Jackson ImmunoResearch Laboratories) was added for 1 h 37° C. After washing, TMB substrate was added and absorbance (630 nm) obtained. Ligand binding assays were performed on serially diluted samples on at least three separate occasions.

Cofactor assays utilized biotinylated human C3b or C4b, human factor I, and varying concentrations of the cofactor proteins in a low salt (25 mM NaCl) buffer. Cleavage fragments were analyzed on 10% reducing gels followed by transfer and Western blotting. Detection was with ExtrAvidin peroxidase conjugate (Sigma-Aldrich). Assays were performed in duplicate on two to four separate occasions.

Decay accelerating assays were performed in microtiter dishes as described (Liszewski, M. K. et al (2006) J. Immunol 176:3725). Briefly, for classical pathway convertase assembly, Ab-coated sheep erythrocytes (Complement Technologies) were incubated with human C1 (1 μg/ml) for 15 min at 30° C. and washed with dextrose gelatin veronal buffer (DGVB⁺⁺). Human C4 (2.2 μg/ml) was added for 15 min at 30° C. and washed. Human C2 (0.25 μg/ml) was added for 4 min at RT and washed. For DAA assessment, 50 μl of inhibitor was added to an equal volume of prepared cells for 10 min at 30° C. Next, 0.5 ml of guinea pig serum (Colorado Serum Company) diluted 1/20 in 40 mM EDTA-GVB was added and incubated for 30 min 37° C. Following centrifugation at 2000×g, the A414 of the supernatant was determined. Assays were performed at least three times with each condition in duplicate.

Heparin-affinity chromatography. Heparin binding was assessed by loading serum free supernatants from transient transfections of SPICE or heparin mutants on a 1-ml HiTrap HP heparin column (GE Healthcare), previously equilibrated with 10 mM phosphate (pH 7.1). The column was washed with 5 ml of equilibration buffer at a flow rate of 1 ml/min, and bound proteins were eluted with a linear gradient from 0 to 2 M NaCl. Fractions were assessed in Western blots.

Generation of murine anti-PICE hybridoma. Mice were immunized with recombinantly produced VCP (Liszewski, M. K. et al (2006) J. Immunol 176:3725) (rVCP) via standard procedures and a mouse with high titer Ab was sacrificed for fusion at the Washington University Hybridoma Center. An assay was developed to select function inhibiting mAbs based upon competitive inhibition of SPICE binding to human C3b by hybridoma supernatants in an ELISA format. In this inhibiting assay, human C3b bearing plates were prepared by coating at 5 μg/ml in PBS overnight. Following inhibiting (PBS, 0.05% Tween-20, 1.0% BSA for 1 h 37° C.), rVCP (1500 ng/ml) and hybridoma supernatant were mixed and pre-incubated for 30 min at 37° C. in low salt buffer (10 mM Tris pH 7.4 and 25 mM NaCl, 0.05% Tween 20, 4% BSA). This mixture was added to the C3b-coated wells and incubated for 1 h at 37° C. Washes were performed in low salt buffer (10 mM Tris pH 7.4, 25 mM NaCl, and 0.05% Tween 20). Binding was detected by the addition of 1:5000 diluted rabbit polyclonal Ab against VCP that cross reacts with SPICE and MOPICE for 1 hr at 37° C. Following washing, a secondary horseradish peroxidase-labeled secondary Ab was incubated for 1 h at 37° C. followed by addition of substrate and measurement of absorbance. Loss of binding was taken as an indication of the presence of a function-inhibiting mAb. Results were compared to screening hybridomas directly against antigen. One hybridoma, KL5.1, was isolated and subcloned. Ascites fluid was prepared and the mAb was purified (performed at Harlan Biosciences). The use of animals in research complied with all required federal guidelines and institutional practices.

### Example 1

### Two SPICE Amino Acids Substituted in VCP Enhance Regulation of Human Complement

In the process of creating SPICE from VCP, three chimeric constructs were evaluated (Table 1) and transiently expressed in Chinese hamster ovary (CHO) cells. Supernatants from these transfectants were concentrated, quantitated (via ELISA), Western blotted and assessed for their ability to regulate activation of human complement. Each of these mutants were expressed and had the expected Mᵣ (see below).

Ligand Binding (C3b). An ELISA was used to profile C3b binding (FIG. 2A). SPICE bound C3b ˜80-fold times more efficiently than VCP. Additionally, mutant R1, which substitutes only two of the SPICE amino acids into VCP (H77 and K120), enhanced C3b-binding capability of VCP by ˜30-fold. This finding is consistent with a previous report that single amino acids substitutions of these residues (VCP-E120K or VCP-Q77H) augmented complement regulatory activity (Sfyroera, G. et al (2005) J. Immunol 174:2143). Generally, for the other two chimeric mutants, C3b binding activity increased as amino acid substitutions from SPICE into VCP were increased.

Ligand Binding (C4b). C4b binding was also assessed by ELISA (FIG. 2B). In contrast to C3b binding, SPICE bound human C4b only 4-fold more efficiently than VCP. Compared to SPICE, chimeras R1, R7 and R9 were similar in their C4b binding profile.

Cofactor Activity (CA) for C3b and C4b. Next CA for C3b and C4b was assessed (Table 2). In all cases, the chimeras were more SPICE-like with a functional enhancement of as much as 100-400 fold compared to VCP. Importantly, mutant R1, carrying only the two SPICE amino acids, H77 and K120, enhanced VCP CA ˜200-fold, indicating the significance of these two amino acids and CCP2 for enhancing SPICE function. These data agree with and extend the observations of Sfyroera et al (J. Immunol (2005) 174:2143) who individually added these SPICE residues to VCP. Both individual mutants had enhanced C3b binding and inhibited C3b deposition in ELISA based assays. These data further illustrate, in addition to the nearly 100-fold increase in C3b binding efficiency of SPICE, that cofactor activity for both C3b and C4b was increased several hundred fold. This occurred despite only a four-fold increase in C4b binding. Overall, these results point to an increase in CA as being key to the increased virulence of SPICE versus VCP for regulating human complement.

Point mutations reveal functional sites. Two sets of point mutations were next evaluated. In the first set, mutations were produced in CCP 2 of SPICE. A peptide in which three of the 11 amino acid differences cluster (i.e., Y98, Y103 and K108) was used (FIG. 1B). Also, this site is homologous to a segment in CCP 2 of CD46 (MCP; membrane cofactor protein), a host complement inhibitor with four CCP repeats (Liszewski, M. K., et al (2000) J. Biol. Chem 275:37692). The three CCP 2 SPICE amino acids were individually mutated to alanines (Y98A, Y103A, and K108A). Following expression and quantitation (as above), they were functionally evaluated (Table 2). Y98A altered C4b binding, C4b cofactor activity and classical pathway DAA, yet was equivalent to wild type relative to C3 interactions. On the other hand, Y103A lost 90% of its C3b cofactor activity and DAA, while maintaining wild type activity for binding to C3b and C4b and for C4b cofactor activity. These data emphasize both the separability of regulatory functions but also a critical role played by this peptide for mediating regulatory activity. Normal C3b binding in the setting of the loss of C3b CA points to a site for a factor I interaction. Loss of DAA by this same residue (Y103A) suggests a role for this site in inhibiting the C3 convertase. Another indication of the fine tuning of regulatory sites was demonstrated by mutant K108A. In this instance, K108A showed a gain of function for ligand binding and DAA. This is consistent with Sfyrorea et al (J. Immunol (2005) 174:2143) who showed that VCP substituted with the corresponding SPICE amino acid at this residue (VCP-E108K) produced enhanced activity. Overall, these mutants support the concept of ligand binding, CA and DAA as being separable regulatory activities.

TABLE 1
Summary of function of SPICE-VCP mutants

| Protein | C3b Cofactor^{a} (ng) | C4b Cofactor^{a} (ng) |
|---|---|---|
| SPICE | 25 | 25 |
| VCP | 10,000 | 10,000 |
| R1 | 50 | 50 |
| R7 | 50 | 50 |
| R9 | 25 | 25 |

^{a}C3b and C4b cofactor activity assays represent the ng needed to cleave 40-60% of the biotinylated ligand in the presence of factor I and monitored by Western blotting and densitometry of bands. For the C3b cofactor assay, comparisons were made based upon the alpha one fragment relative to the beta-chain. For the C4b cofactor, comparisons were made based on loss of alpha one chain relative to the gamma-chain fragment of C4b. Chimeras enhanced cofactor activity 200-400 fold. Data represent mean +/− SEM from three to four experiments.

TABLE 2
Complement regulatory profile of point mutations in CCP2 of SPICE^{a}

| | | | C3b | C4b | |
|---|---|---|---|---|---|
| SPICE | | C4b | Cofactor | Cofactor | |
| MUTANT | C3b Binding | Binding | Activity | Activity | DAA (CP) |
| Y98A | 98 ± 4 | 61 ± 3 | 100 ± 4 | 50 ± 2 | 60 ± 8 |
| Y103A | 96 ± 5 | 99 ± 3 | 10 ± 1 | 96 ± 3 | 10 ± 1 |
| Y108A | 184 ± 18 | 180 ± 19 | 119 ± 12 | 112 ± 13 | 172 ± 17 |

^{a}Data are presented as per cent activity of wild-type SPICE (mutants and SPICE expressed in CHO cells). C3b/C4b binding and cofactor assay conditions described in Table 3. For DAA for the classical pathway C3 convertase, supernatants of SPICE or mutant (20 ng) were incubated 10 min with prepared sheep erythrocytes. Guinea pig serum was then added for 30 min. DAA is described relative to SPICE and represents percentage of inhibition as calculated from comparisons to conditions without added inhibitor. Data represent mean ± SEM for three or four experiments.

### Example 2

### Electrophoretic Mobility of VCP and SPICE is Influenced by Residue 131

Although VCP and SPICE each contain 244 residues with a similar mass (VCP mol wt 26,745; SPICE mol wt 26,863), SPICE migrates with a faster Mᵣ on gels (nonreducing SDS-PAGE). Both the monomers and dimers show this pattern (Liszewski, M. K. et al (2006) J. Immunol 176:3725). It was noted that SPICE-VCP chimeras lacking L131 migrated with an Mᵣ similar to VCP, while others containing L131 migrated with an Mᵣ similar to SPICE (R5, R6, R7, and R9). Consequently, we substituted the L131 residue of SPICE to the homologous VCP residue S131 (i.e., SPICE mutant L131S) and transiently expressed this protein in these CHO cells. Western blot analysis of the transfectant media revealed that monomeric and dimeric SPICE-L131S migrate with a Mᵣ similar to VCP and not SPICE (FIG. 3A). Thus, L131S is responsible for the majority of Mᵣ difference between the two proteins.

Next the functional impact of this mutation was assessed (FIG. 3B). L131S demonstrated a specific loss (˜90%) of C3b CA. In contrast, it retained an activity profile similar to wild-type SPICE with respect to C3b/C4b binding, C4b CA and classical pathway DAA. SPICE residue L131 likely produces a conformational change in SPICE that enhances cofactor activity against human C3b. This is consistent with the ˜100× greater cofactor activity of SPICE as compared to VCP and with earlier findings (Table 2) that individual amino acids can mediate distinct inhibitory activities. Thus, alteration of a single amino acid in SPICE nearly abrogates its ability to degrade human C3b.

### Example 3

### Validation of Heparin Binding Sites

To examine the influence of three putative heparin binding sites in poxviruses, three proposed heparin binding sites of SPICE were individually mutated to create heparin mutants HS1, HS2, and HS3 (FIG. 1). These were transiently expressed in CHO cells, and the supernatants were chromatographed over a heparin column. Following elution under increasing salt concentrations, fractions were monitored via Western blotting using a cross-reacting anti-VCP Ab. As summarized in Table 3, mutation of each of the individual sites diminished the ability of the protein to bind to heparin as compared to wild type. However, mutation of the second site decreased its ability to bind to heparin to a greater extent than HS1 and HS3. The peak elutions of the mutants are summarized in Table 3. All eluted at a lower ionic strength than wild type SPICE. Thus, each of these three sites contributes to the ability of SPICE (and likely other PICES) to bind heparin, yet HS2 has more of an impact. As suggested previously, the overall positive charge of SPICE likely also contributes to its ability to bind heparin.

TABLE 3
Heparin binding and complement regulatory profile of SPICE HS mutants^{a}

| | | | | | C4b |
|---|---|---|---|---|---|
| | Peak Elution^{b} | C3b Binding^{c} | C4b Binding^{c} | C3b Cofactor^{d} | Cofactor^{d} |
| Protein | (mM NaC1) | (% Wild type) | (% Wild type) | (% Wild type) | (% Wild type) |
| SPICE | 447 ± 5 | 100 ± 5 | 100 ± 4 | 100 ± 7 | 100 ± 8 |
| HS1 | 384 ± 3 | 80 ± 2 | 46 ± 2 | 42 ± 4 | 48 ± 3 |
| HS2 | 328 ± 3 | 116 ± 3 | 87 ± 4 | 54 ± 5 | 88 ± 6 |
| HS3 | 388 ± 4 | 59 ± 4 | 95 ± 6 | 54 ± 2 | 76 ± 8 |
| HS1-2-3 | 239 ± 6 | 11 ± 1 | 4 ± 1 | 8 ± 5 | None detected |

^{a}SPICE with mutated heparin binding sites were expressed in 293T cells and the supernatants were evaluated.
^{b}SPICE supernatants were chromatographed over a heparin column, eluted with increasing salt concentrations and fractions monitored via Western blot with a cross-reacting polyclonal Ab to VCP. Peak elution of monomer represents the mean ± SEM of three or four experiments.
^{c}C3b and C4b binding were performed in an ELISA format in which human C3b or C4b were adsorbed to microtiter plates. SPICE and mutant supernatants (10 ng/ml) were applied and then detected with a polyclonal antibody. SPICE binding was set at 100%. Data are % of wild-type SPICE and represent mean ± SEM for four experiments.
^{d}For the C3b and C4b cofactor assays, biotinylated C3b or C4b was incubated with human factor I and transfectant supernatants (1.7 ng/ml of SPICE or mutant for C3b assays and 6.7 ng/ml for C4b assays) and evaluated on Western blots using HRP-Extravidin. For C3b cofactor activity, comparisons were made based upon the loss of the α′ chain and development of the α1 cleavage fragment. For C4b cofactor activity, comparisons were made based on density of the α′ band relative to the β chain. Data are % of wild-type SPICE and represent mean ± SEM for four experiments.

### Example 4

### Heparin Sites Impact Complement Regulation

Next the HS mutants were assessed for their impact on complement regulatory activity. Previous studies have implicated SPICE CCP1 in complement inhibition (Rosengard, A. M., et al (1999) Mol. Immunol 36:685; Mullick, J. et al (2005) J. Virol. 79:12382) and suggested residues K12, K14, K41, K43, K64, R65 and R66 were important for C3b/C4b interactions (Ganesh, V. K. et al (2004) Proc. Natl. Acad. Sci. 101:8924). To further address this possibility, we characterized the ability of these mutants to regulate complement. As summarized in Table 3, mutant HS1 retained ligand binding and cofactor activity. HS2 had reduced binding and a similar reduction of cofactor activity for C4b. HS3 lost ligand binding and cofactor activity. These data suggest that the sites for putative heparin binding are also involved in complement regulation.

Mutant HS3 is part of an epitope in CCP 1 for mAb to SPICE which blocks regulatory activity. In addition to further defining the active sites in SPICE, it was desired to develop mAbs that inhibit SPICE activity.

### Example 5

### Developing an Anti-PICE Antibody

To select a function-inhibiting mAb, an ELISA was employed to select hybridoma supernatants that could competitively inhibit SPICE binding to human C3b (see Methods). mAb KL 5.1 bound SPICE, MOPICE and VCP equivalently across a range of dilutions (FIG. 4A). Additionally, the Ab bound equally well if the poxviral protein was produced in mammalian, yeast or E. coli expression systems. mAb KL5.1 inhibited DAA of SPICE and VCP in a dose-dependent manner (FIG. 4B). In cofactor assays, mAb KL5.1 inhibited C3b cofactor and C4b activity for all three PICES (FIG. 4C). Thus, mAb KL5.1 inhibits both cofactor and decay accelerating activities, suggesting a common functional domain among the PICES for these activities.

To map the binding site, we initially assessed KL5.1 binding to the mutants. In Western blotting analysis, it did not bind to the HS3 mutant suggesting that this site may comprise or modulate the epitope. This finding also validates our method for developing function-inhibiting PICE mAbs and correlates with our earlier finding that this mutant lacked the ability to regulate complement. This mAb has also been successfully employed as a capture Ab reagent in an ELISA to quantify PICES [detection range from 0.1 to 2 ng/mL].

### Materials and Methods for Examples 6-11

Generation of stable lines expressing SPICE-TM. Unless otherwise noted, Chinese hamster ovary cells (CHO) were the CHO-K1 cell line from American Type Culture Collection (Manassas, Va.). Generation of the MCP 3-10 CHO cell line was previously described (18). To prepare transmembrane SPICE expressed in CHO, CCPs 1-4 were generated by PCR from the previously described SPICE cDNA (4) using the following primers: 5′ GCGGATCCGGAATGGGAATGAAGGTGGAGAGCGTG 3′ (SEQ ID NO:1) and 5′ CCGGAATTCGCGTACACATTTTGGAAGTTC 3′ (SEQ ID NO:2). It was subsequently cloned into the BamH1 and EcoR1 sites of pcDNA3 (Invitrogen). The resulting plasmid was digested with EcoR1 and Not1 and ligated with an MCP-BC1 fragment containing the juxtamembraneous 10 amino acid domain, transmembrane domain and cytoplasmic tail generated from the template MCP-BC1 using the following primers: 5′ CCGGAATTCGGATATCCTAAACCTGAGGA 3′(SEQ ID NO:3) and 5′ATAAGAATGCGGCCGCTTAGCATATTCAGCTCCACCATC 3′(SEQ ID NO:4).

Pvu1 linearized DNA was then transfected into CHO cells using FUGENE-6 (Roche), according to the manufacturer's recommendations. Cells were maintained in Ham's F12 with 10% heat inactivated FBS. After 48 h, G418 was added at an activity concentration of 0.5 mg/ml. G418 resistant pools, labeled with a polyclonal Ab that recognizes SPICE (4), were sorted according to expression level. Single cells were deposited onto a 96-well plate using a MoFlo high speed flow cytometer (DAKO Cytomation). A stable line was selected for SPICE surface expression by FACS and utilized in these studies (clone H3).

Cell lines. Cell lines used to assess SPICE binding were obtained from the Washington University Tissue Culture Support Center. The HeLa epithelial cell line was grown in Dulbecco's Modified Eagles Medium, 2 mM L-glutamine and 10% FBS; HepG2 epithelial cell line was grown in MEM plus Earle's salts with 2 mM L-glutamine and 10% FBS; the HaCat, keratinocyte line, was grown in Dulbecco's Modified Eagle's Medium with 8 mM L-glutamine, 50 mM HEPES and 10% FBS; the HMEC endothelial cell line was grown in MCDB 131 (Invitrogen) supplemented with 10% FBS, 2 mM L-glutamine, 0.3% NaHCO₃, 1 μg/ml hydrocortisone (Sigma), 10 ng/ml epidermal growth factor (Sigma); IMR-90, a fibroblast cell line, was grown in Dulbecco's Modified Eagles Medium, 2 mM L-glutamine and 10% FBS. CHO K1, 745, and M1 cell lines were grown in Ham's F12 supplemented with 10% heat inactivated FBS and 2 mM L-glutamine. These cell lines have been previously described in our laboratory (19-24). Media were supplemented with 100 units/ml penicillin G and 100 μg/ml streptomycin sulfate.

Isolation of human peripheral blood subsets. Primary human peripheral blood mononuclear cells (PBMC) were isolated from the buffy coats of healthy volunteers by Ficoll-Hypaque (Pharmacia) density gradient centrifugation per manufacturer's protocol. Human CD14+ monocytes, CD19+ B cells and CD4+ T lymphocytes were purified from the PBMC by positive selection using antibody-coated magnetic beads (Miltenyi Biotec) per manufacturer's direction. Red blood cells were obtained from whole blood from a normal volunteer via established protocols (25).

SPICE binding to cells and evaluation by flow cytometry. Cells were obtained by treatment either with 0.05% trypsin/0.53 mM EDTA or 4 mM EDTA (both from Mediatech) and followed by washes with PBS/1% FBS. Typically, 1×10⁶ cells were mixed with 20 μg of SPICE in a total volume of 100 μl and incubated at 30° C. with gentle shaking in an Eppendorf Thermomixer for 30 min. Following incubation, cells were placed on ice and washed with PBS/1% FBS. SPICE was detected by flow cytometry using a previously prepared rabbit polyclonal Ab against VCP (4) followed by incubation with a FITC-donkey anti-rabbit IgG secondary Ab (Sigma). Cells were fixed in 0.5% paraformaldehyde in PBS.

Sodium chlorate treatment of cultured cells. Sulfation was inhibited by sodium chlorate treatment (26). CHO cells were cultured in sulfate-free Ham's F12 medium (Washington University Tissue Culture Support Center) supplemented with 10% dialyzed FBS (Fisher Scientific) containing different concentrations (1-25 mM) sodium chlorate. After overnight culture, cells were processed for SPICE binding as described above.

Production of recombinant SPICE in E. coli. Using SPICE cDNA (4) as a template, the coding sequence of SPICE was generated by PCR using the following oligos: 5′CGCGGATCCATGTGCTGTACTATTCCGTCAC 3′ (SEQ ID NO:5) and 5′ ATAAGAATGCGGCCGCTTATTTTGGAAGTT 3′ (SEQ ID NO:6). The resulting PCR fragment was ligated into the BamH1 and Not1 sites of pET28a(+)-2, a derivative generated in our laboratory of pET28a(+) (EMD/Novagen). For recombinant protein production a method was developed (modified from (4)). First, 25 ml of an overnight culture of E. coli containing the construct was inoculated into 500 ml of LB containing kanamycin (30 μg/mi) and chloramphenicol (34 μg/ml) and grown to an OD600 of 0.6-0.8 followed by induction with 1 mM IPTG at 37° C. for an additional 3-5 hr. Cells were harvested and pellets were frozen at −80° C. until needed.

For inclusion body protein purification, pellets were thawed and resuspended in 50 ml of Solution Buffer (50 mM Tris, pH 8.0, 25% sucrose, 1 mM EDTA, 0.01% NaN₃, and 10 mM DTT) to which 0.8 ml of freshly prepared 50 mg/ml lysozyme (Sigma), 1250 units of benzonase nuclease (Novagen), and 1 ml of 1 M MgCl₂ were added. An equal volume of Lysis Buffer (50 mM Tris, pH 8.0, 1% Triton X-100, 0.1 M NaCl, 0.01% NaN₃, and 10 mM DTT) was added and the solution was stirred gently at room temperature for one h. After cooling, the suspension was sonicated with three 15s bursts (Fisher Scientific Model 500 Sonic Dismembrator) at 50% amplitude followed by the addition of 5 ml of 0.5 M EDTA. The lysate was then centrifuged at 6000 g for 30 min at 4° C. The resulting inclusion body pellet was washed (50 mM Tris, pH 8.0, 0.5% Triton X-100, 0.1 M NaCl, 1 mM EDTA, 0.01% NaN₃, and 1 mM DTT) followed by a second wash with the same buffer, but without Triton X-100.

For solubilization of the inclusion bodies, the pellet was resuspended in 6 M guanidine HCl, 10 mM Tris pH 8.0, and 20 mM β-mercaptoethanol and centrifuged at 14,000 g for 10 min. A second high speed centrifugation at 100,000 g for 30 min at 4° C. was performed to remove any insoluble material.

For protein refolding, solubilized inclusion body protein was added dropwise in three injections over 36 h at a final concentration of 10-100 μg/ml in refolding buffer (100 mM Trizma Base, 400 mM L-arginine-HCl (Sigma), 2 mM EDTA, 0.02 M ethanolamine, 0.5 mM oxidized glutathione (Sigma), and 5 mM reduced glutathione (Sigma). The refolding solution was concentrated in a Millipore Stirred Filtration Cell followed by buffer exchange with 20 mM Tris, pH 8.0.

C3b and C4b binding. An ELISA format was used for ligand binding as described (4). Briefly, human C3b or C4b (Complement Technologies) was coated overnight at 4° C. on wells at 5 μg/ml in PBS followed by inhibiting for 1 h at 37° C. with 1% BSA, 0.1% Tween 20 in PBS. Proteins were diluted in low salt ELISA buffer (10 mM Tris, pH 7.2, 25 mM sodium chloride, 0.05% Tween 20, 4% BSA, and 0.25% Nonidet P-40) and incubated for 1.5 h at 37° C. (4). Rabbit anti-VCP Ab ( 1/5000) in low salt buffer was then added for 1 h at 37° C. Following washing, a peroxidase-coupled donkey anti-rabbit IgG was added, and OD of the TMB substrate was determined. Binding assays were performed employing serially diluted samples on at least four separate occasions.

Initiation of Complement Activation. The standard procedure for the initiation of the complement pathways has been reported (27). Briefly, CHO cells are grown to ˜70% confluency and collected by trypsinization into 1% FCS-PBS. Sensitizing antibody was an IgG prepared from rabbits injected with whole CHO cells (Harlan). The Ab was added to cells and the mixture incubated for 30 min at 4° C. Following two washes with 1% FCS-PBS, 100 μl of C8-deficient (C8d) serum (donated by P. Densen, University of Iowa, Iowa City, Iowa) in gelatin veronal-buffered saline (GVB⁺⁺) (Complement Technologies) was added. To inhibit the classical pathway, gelatin veronal-buffered saline (GVB⁰) (Complement Technologies) was used with added 10 mM EGTA and 7 mM magnesium chloride (Mg²⁺-EGTA). Cells were harvested at indicated time points and washed twice in PBS containing 1% FCS before C4 and C3 fragment analysis.

FACS analysis of complement fragment deposition. This procedure has been previously described (27). Briefly, following complement deposition and two washes, murine mAbs to the human complement component fragments C3c, C3d, C4c, or C4d (Quidel) were added (5 mg/ml). After a 30 min incubation at 4° C., FITC-conjugated goat anti-mouse IgG (Sigma) was added for 30 min at 4° C. Cells were fixed with 0.5% paraformaldehyde and analyzed on a BD Biosciences FACSCalibur system (BD Biosciences).

Soluble GAG Binding Assays. SPICE (10 μg) was preincubated with soluble GAGs (5 μg/ml) in a total volume of 100 μl in PBS at room temperature for 20 min. After preincubation, the mixture was added to 5×10⁵ CHO cells (harvested from flasks using 4 mM EDTA) in a total volume of 100 μl and incubated at 30° C. for 30 min with gentle mixing. The cells were washed with PBS. SPICE was detected by flow cytometry using a rabbit polyclonal Ab as described above.

### Example 6

### Preparation and Isolation of Clones

To analyze human complement regulatory activity by membrane-bound SPICE, we fused the sequence for the transmembrane domain of the human complement regulator membrane cofactor protein (MCP) onto SPICE to create SPICE-TM (FIG. 7A). Stably transfected Chinese hamster ovary (CHO) clones expressing SPICE-TM were isolated and further evaluated. A SPICE clone that had the equivalent expression (˜25,000/cell) to an MCP clone, MCP-3-10 (27) was selected (FIGS. 7B and 7C). Flow cytometry with mAbs to MCP and SPICE and employing the same secondary Ab further established that the expression levels were similar. The Western blot showed the expected Mᵣ (FIG. 7C) for SPICE-TM. Approximately 10% of the protein was expressed as a dimer as has been previously described (4).

### Example 7

### SPICE-TM Regulates Human Complement on CHO Cells

We assessed the ability of SPICE-TM to regulate human complement on the cell surface and profiled its activity relative to that of the native regulator MCP. Using the clones described in FIG. 7, we activated the alternative pathway of complement using a previously described model system (27, 28). In this “complement challenge” design, rabbit anti-CHO Ab is used to sensitize cells followed by exposure to a source of nonlytic complement (10% C8-deficient serum) diluted in a buffer (Mg++GVB) that allows for only alternative pathway activation. Quantity of C3b deposited was then assessed by FACS with a mAb (anti-C3d) that recognizes cleavage fragments of C3 containing this fragment.

FIG. 8A demonstrates that SPICE-TM inhibits C3 fragment deposition similarly to the MCP-expressing clone (shown in FIG. 7). Following AP activation, large quantities of C3 fragments deposit on CHO cells that do not express a regulatory protein. In contrast, both SPICE-TM and MCP clones, carrying approximately equal copy number of each inhibitor, decrease C3 deposition by ≧90% [MFIs of 22 (MCP), 31 (SPICE-TM) vs 307 (CHO)]. Thus, transmembrane SPICE regulates the alternative pathway with similar efficiency to that of the native regulator MCP. Also, the regulation is primarily mediated by cofactor activity since MCP has no (29) and SPICE barely detectable DAA for the alternative pathway (4).

### Example 8

### mAb KL5.1 Inhibits SPICE Complement Regulatory Function on Cells

In a report to be published elsewhere, we describe a mAb that binds soluble SPICE and inhibits its C3b and C4b binding and cofactor activity (30). To assess its ability to inhibit the function of cell-bound SPICE, we pre-incubated this mAb with the SPICE-TM clone and then challenged the cells as described above. This mAb abrogated SPICE-TM complement regulatory activity (FIG. 8B). Its profile relative to C3d deposition was similar to CHO cells lacking a regulator. An isogenic IgG control did not reduce SPICE's inhibitory activity (‘Neg’ profile, FIG. 8B).

### Example 9

### Expression and Characterization of SPICE in E. coli

We next asked if soluble SPICE could attach to cells and regulate complement activation. Previous studies established that SPICE binds heparin, providing a possible mechanism for its attachment to membranes and extracellular matrices (4).

To assess whether SPICE can bind to cells, we prepared the soluble, recombinant protein in an E. coli expression system. SPICE migrated predominantly as a single band on reducing (R) and nonreducing (NR) gels (SDS-PAGE) as assessed by Coomassie blue staining (FIG. 9A) and Western blotting (FIG. 9B). The slower Mᵣ on reducing gels is characteristic of CCP containing proteins as each module contains two disulfide bridges (31). Because this protein was produced in E. coli and requires refolding from inclusion bodies, as part of its characterization we also performed functional analyses. SPICE produced by E. coli bound human C3b/C4b analogous to what we observed with mammalian expression systems (FIG. 9C) (4) and had comparable cofactor activity for C3b. Also, the absence of disulfide dependent dimer formation (compare to FIG. 7C) is consistent with expression in the E. coli system (4, 6).

To assess binding of SPICE to CHO cells, we incubated SPICE (50-400 μg/ml) with 5×10⁵ cells and monitored attachment with a cross-reacting rabbit polyclonal anti-VCP Ab (FIG. 10). Increasing amounts of SPICE led to greater quantities being deposited on the surface.

We next characterized the ability of SPICE to bind a variety of human cell lines (FIG. 11). SPICE attached to all six human cell lines shown (FIG. 11A), although there was minimal binding to HMEC. On human peripheral blood cells, SPICE bound to B cells and monocytes to a similar extent, to a lesser degree to T cells and, minimally, to red blood cells (FIG. 11B). These data suggest that different types of heparin or heparin-like constituents on a given cell membrane influence the ability of SPICE to attach to cells.

### Example 10

### SPICE Binding to Cells is GAG Dependent and is Inhibited by Heparin and Chondroitin Sulfate-E

To determine if the binding of SPICE is dependent on glycosaminoglycans, we compared the binding of SPICE on wild-type CHO cells with two GAG-deficient CHO cell lines (FIG. 12A). The cell line 745-CHO lacks xylosyltransferase, the enzyme required for biosynthesis of both heparan sulfate (HS) and chondroitin sulfate (CS) (19). Cell line M1-CHO lacks surface heparan sulfate (23). Following incubation with cells, SPICE bound to wild-type CHO and M1-CHO but had reduced (˜90%) binding activity to 745-CHO (FIG. 6A). These results suggest that the mechanism for SPICE binding is likely to be GAG-dependent.

To define the nature of the GAGs responsible for SPICE binding, we performed competitive inhibition binding assays with soluble GAGs (FIG. 12B). There was significant inhibition by heparin (HP) (75%) and chondroitin sulfate-E (CS-E) (90%) and a lesser degree of inhibition by CS-D (54%) and CS-C (44%). CS-A and CS-B did not modulate SPICE binding. Taken together, these results suggest that SPICE interacts strongly with heparin CS-E and less avidly with CS-C and -D. Interestingly, CS-E is enriched in disulfated disaccharides and is unique compared to other CS in that two sulfates are present in the same GaINAc residue. Therefore, clustered sulfates rather than net negative charge on the disaccharide backbone may facilitate SPICE binding.

To further establish the necessity of clustered sulfates, we treated CHO cells with the reversible sulfation inhibitor, sodium chlorate (26) (FIG. 13). Binding of SPICE was decreased by ˜90% following the addition of 1-25 mM sodium chlorate. This inhibition was completely reversed by the exogenous addition of sodium sulfate and sodium chlorate (25 mM each). Thus these studies establish that sulfated GAGs are ligands involved in binding of SPICE to cells.

### Example 11

### rSPICE and Complement Activation

To determine if SPICE binding to cells via GAGs inhibits complement activation, we sensitized cells with CHO Abs to activate the classical or alternative pathways (32, #2459). Relative to classical pathway activation, SPICE decreased C3b deposition by 50% [FIG. 14A, compare dark line of SPICE (MFI 2,106) to the shaded area of CHO (MFI 4,350)]. This result is consistent with SPICE possessing DAA for the classical pathway convertase (4) and comparable to DAF's activity in this same experimental system (33). As expected, MCP did not reduce C3b deposition (MFI 4290), which is consistent with previous findings that MCP controls the alternative but not classical pathway on the cell surface (27). For alternative pathway activation (FIG. 14B), both regulators decreased C3b deposition to a similar extent (96% for SPICE, 94% for MCP; MFIs for CHO, SPICE, and MCP were 630, 28 and 39, respectively). Of note, there was a similar degree of inhibition of SPICE and MCP, yet SPICE was present on the cell surface at 5-10 fold less than the level of MCP (FIG. 14C). Table 4 compares the percent inhibition of C3b deposition on CHO cells expressing SPICE, SPICE-TM or MCP. All three decrease deposition similarly. From these data, we conclude that SPICE is an efficient inhibitor of the alternative pathway especially when attached to cells via GAGs. Additionally, since SPICE has very little DAA for the alternative pathway (4), these results further suggest that greater cofactor activity or enhanced mobility of SPICE attached via GAGs translates into more efficient inhibition of the alternative pathway.

TABLE 4
Comparison of inhibition of alternative pathway C3b deposition by
SPICE attached to CHO cells via GAG or transmembrane anchor

| Protein/Cell Line* | % Inhibition of C3b Deposition⁺ |
|---|---|
| MCP | 89 ± 3 |
| SPICE-TM | 87 ± 3 |
| SPICE | 85 ± 2 |

*SPICE is the recombinant soluble purified protein expressed in E. coli. SPICE-TM is a CHO cell clone expressing ~25,000 SPICE/cell that is a recombinant protein bearing the transmembrane domain of MCP. MCP is a CHO clone bearing ~25,000 MCP/cell.
⁺C3b deposition methods as described above. Data represent the mean ± SEM from four to six experiments done in duplicate.

SPICE, similar to MCP, cleaves C4b after it is deposited on a cell by serving as a cofactor for its factor I-mediated cleavage into fragments C4c and C4d (4-6). Since soluble SPICE has C4b cofactor activity (4, 5), we asked whether SPICE inactivates C4b deposited on cells following complement activation (FIG. 15). C4b degradation by SPICE and MCP was monitored utilizing mAbs to C4d. This fragment remains covalently bound to cells following cleavage of C4b and release of fragment C4c. We found that C4d and C4c levels are similar on control cells indicating that C4b is present on these cells lacking a cofactor protein for C4b degradation (FIG. 15A). However, in the presence of SPICE, C4c levels are progressively reduced over a 45 min period (FIG. 15B). MCP also showed increased C4b cleavage from 15 to 45 min consistent with previous findings (34). However, SPICE cleaves C4b more quickly and to a greater degree (despite being present at 5-10 fold less than MCP, see FIG. 15C). The percent C4 cleavage of SPICE versus MCP at 15 and 45 min is 57% and 82% for SPICE versus 13% and 58% for MCP, respectively (see MFIs in FIG. 14 legend).

From these data, we conclude that SPICE is an efficient cofactor for the factor I mediated degradation of human C4b deposited on cells. Further, SPICE is more efficient in this regard than MCP, perhaps secondary to enhanced mobility.

### Example 12

### KL5.1 mAb Detects Infections Virions and Decreases the CA of Poxvirus Complement Inhibitors

Western blots were performed and show that KL5.1 mAb detects PICE proteins deposited on infectious virions. (FIG. 5) Mammalian cells were infected with the vaccinia virus. Intracellular mature virus (IMV) and extracellular enveloped virus (EEV) particles were isolated and lysed. Following SDS-PAGE, the gel was probed (Western blot) with (FIG. 5A) polyclonal rabbit-anti-VCP Ab, or (FIG. 5B) KL 5.1 mAb. As indicated, the number of plaque forming units (pfu) per lane applied was 3.25, 6.5, and 65×10⁴.

FIG. 6 depicts western blots showing that monoclonal Ab KL5.1 inhibits cofactor activity of SPICE, MOPICE and VCP. Biotinylated ligand (C3b or C4b) was incubated with factor I and the supernatants from CHO transfectants. Following electrophoresis on 10% SDS-PAG, Western blofting was performed using HRP-avidin. Loss of the α′ chain and development of α₁ cleavage fragments were monitored. The α₂ fragments were visible after longer exposure times. The mAb inhibits ability of SPICE to serve as a cofactor for C3b cleavage. (FIG. 6A) The mAb shows partial inhibiting of MOPICE (FIG. 6B) and VCP (FIG. 6C). mAb KL5.1 inhibits CA for C4b for SPICE (FIG. 6D), MOPICE (FIG. 6E), and VCP (FIG. 6F).

### Example 13

### mAb KL5.1 may be Used to Detect Poxvirus Complement Inhibitors and Decrease Poxvirus Complement Inhibitor Activity

The mAb KL5.1 may be used as a capture antibody in an ELISA to detect and quantify PICES. (FIG. 16) The mAb was adsorbed to microtiter wells and dilutions of VCP added followed by detection with rabbit polyclonal anti-VCP antibody (Ab) and HRP anti-globulin Ab. Negative control (background) was subtracted. Lower limits of detection were 0.5 to 1.0 ng/ml.

The mAb KL5.1 may also be used to detect PICES in a Western blot. (FIG. 17) PICE samples were electrophoresed on 12% SDS-PAG, transferred to nitrocellulose, and probed with either the rabbit anti-VCP Ab (lanes 1-5) or KL5.1 mAb.

FIG. 18 depicts a Western blot and a graph showing that mAb KL5.1 inhibits C3b cofactor activity. FIG. 18A demonstrates the decrease in CA by mAb KL5.1. FIG. 18B demonstrates that the mAb KL5.1 inhibits decay accelerating activity (DAA).

### REFERENCES

1. Reed, K. D., Melski, J. W., Graham, M. B., Regnery, R. L., Sotir, M. J., Wegner, M. V., Kazmierczak, J. J., Stratman, E. J., Li, Y., Fairley, J. A., Swain, G. R., Olson, V. A., Sargent, E. K., Kehl, S. C., Frace, M. A., Kline, R., Foldy, S. L., Davis, J. P. & Damon, I. K. (2004) The detection of monkeypox in humans in the Western Hemisphere, New England Journal of Medicine 350:342-50.
2. Di Giulio, D. B. & Eckburg, P. B. (2004) Human monkeypox: an emerging zoonosis. Lancet Infect Dis 4:15-25.
3. Jezek, Z., Marennikova, S. S., Mutumbo, M., Nakano, J. H., Paluku, K. M. & Szczeniowski, M. (1986) Human monkeypox: a study of 2,510 contacts of 214 patients. Journal of Infectious Diseases 154:551-555.
4. Liszewski, M. K., Leung, M. K., Hauhart, R., Buller, R. M., Bertram, P., Wang, X., Rosengard, A. M., Kotwal, G. J. & Atkinson, J. P. (2006) Structure and regulatory profile of the monkeypox inhibitor of complement: comparison to homologs in vaccinia and variola and evidence for dimer formation. J Immunol 176:3725-34.
5. Rosengard, A. M., Liu, Y., Nie, Z. & Jimenez, R. (2002) Variola virus immune evasion design: expression of a highly efficient inhibitor of human complement. Proc Natl Acad Sci USA 99:8808-13.
6. Sfyroera, G., Katragadda, M., Morikis, D., Isaacs, S. N. & Lambris, J. D. (2005) Electrostatic modeling predicts the activities of orthopoxvirus complement control proteins. Journal of Immunology 174:2143-51.
7. Yadav, V. N., Pyaram, K., Mullick, J. & Sahu, A. (2008) Identification of hot spots in the variola virus complement inhibitor (SPICE) for human complement regulation. J Virol.
8. Ganesh, V. K., Smith, S. A., Kotwal, G. J. & Murthy, K. H. M. (2004) Structure of vaccinia complement protein in complex with heparin and potential implications for complement regulation. Proceedings of the National Academy of Sciences of the United States of America 101:8924-8929.
9. Murthy, K. H., Smith, S. A., Ganesh, V. K., Judge, K. W., Mullin, N., Barlow, P. N., Ogata, C. M. & Kotwal, G. J. (2001) Crystal structure of a complement control protein that regulates both pathways of complement activation and binds heparan sulfate proteoglycans. Cell 104:301-311.
10. Smith, S. A., Mullin, N. P., Parkinson, J., Shchelkunov, S. N., Totmenin, A. V., Loparev, V. N., Srisatjaluk, R., Reynolds, D. N., Keeling, K. L., Justus, D. E., Barlow, P. N. & Kotwal, G. J. (2000) Conserved surface-exposed K/R-X-K/R motifs and net positive charge on poxvirus complement control proteins serve as putative heparin binding sites and contribute to inhibition of molecular interactions with human endothelial cells: a novel mechanism for evasion of host defense. Journal of Virology 74:5659-66.
11. Meri, S. & Pangburn, M. K. (1994) Regulation of alternative pathway complement activation by glycosaminoglycans: specificity of the polyanion binding site on factor H. Biochemical & Biophysical Research Communications 198:52-9.
12. Girgis, N. M., Dehaven, B. C., Fan, X., Viner, K. M., Shamim, M. & Isaacs, S. N. (2008) Cell surface expression of the vaccinia virus complement control protein is mediated by interaction with the viral A56 protein and protects infected cells from complement attack. J Virol.
13. Fauci, A. S. & Challberg, M. D. (2005) Host-based antipoxvirus therapeutic strategies: turning the tables. J Clin Invest 115:231-3.
14. Harrison, S. C., Alberts, B., Ehrenfeld, E., Enquist, L., Fineberg, H., McKnight, S. L., Moss, B., O'Donnell, M., Ploegh, H., Schmid, S. L., Walter, K. P. & Theriot, J. (2004) Discovery of antivirals against smallpox. Proceedings of the National Academy of Sciences of the United States of America Early Edition: 1-15.
15. Isaacs, S. N., Argyropoulos, E., Sfyroera, G., Mohammad, S. & Lambris, J. D. (2003) Restoration of complement-enhanced neutralization of vaccinia virus virions by novel monoclonal antibodies raised against the vaccinia virus complement control protein. Journal of Virology 77:8256-62.
16. Isaacs, S. N., Kotwal, G. J. & Moss, B. (1992) Vaccinia virus complement-control protein prevents antibody-dependent complement-enhanced neutralization of infectivity and contributes to virulence. Proc Natl Acad Sci USA 89:628-32.
17. Kotwal, G. J., Isaacs, S. N., McKenzie, R., Frank, M. M. & Moss, B. (1990) Inhibition of the complement cascade by the major secretory protein of vaccinia virus. Science 250:827-30.
18. Tortorella, D., Gewurz, B. E., Furman, M. H., Schust, D. J. & Ploegh, H. L. (2000) Viral subversion of the immune system. Annu Rev Immunol 18:861-926.
19. Esko, J. D., Stewart, T. E. & Taylor, W. H. (1985) Animal cell mutants defective in glycosaminoglycan biosynthesis. Proc Natl Acad Sci USA 82:3197-201.
20. Zhang, L., Beeler, D. L., Lawrence, R., Lech, M., Liu, J., Davis, J. C., Shriver, Z., Sasisekharan, R. & Rosenberg, R. D. (2001) 6-O-sulfotransferase-1 represents a critical enzyme in the anticoagulant heparan sulfate biosynthetic pathway. J Biol Chem 276:42311-21.
21. Xia, G., Chen, J., Tiwari, V., Ju, W., Li, J. P., Malmstrom, A., Shukla, D. & Liu, J. (2002) Heparan sulfate 3-O-sulfotransferase isoform 5 generates both an antithrombin-binding site and an entry receptor for herpes simplex virus, type 1. J Biol Chem 277:37912-9.
22. Duncan, M. B., Chen, J., Krise, J. P. & Liu, J. (2004) The biosynthesis of anticoagulant heparan sulfate by the heparan sulfate 3-O-sulfotransferase isoform 5. Biochim Biophys Acta 1671:34-43.
23. Broekelmann, T. J., Kozel, B. A., Ishibashi, H., Werneck, C. C., Keeley, F. W., Zhang, L. & Mecham, R. P. (2005) Tropoelastin interacts with cell-surface glycosaminoglycans via its COOH-terminal domain. J Biol Chem 280:40939-47.
24. Zhang, L., Lawrence, R., Frazier, B. A. & Esko, J. D. (2006) CHO glycosylation mutants: proteoglycans. Methods Enzymol 416:205-21.
25. Avirutnan, P., Zhang, L., Punyadee, N., Manuyakorn, A., Puttikhunt, C., Kasinrerk, W., Malasit, P., Atkinson, J. P. & Diamond, M. S. (2007) Secreted NS1 of dengue virus attaches to the surface of cells via interactions with heparan sulfate and chondroitin sulfate E. PLOS Pathogens 3:1798-1812.
26. Baeuerle, P. A. & Huttner, W. B. (1986) Chlorate—a potent inhibitor of protein sulfation in intact cells. Biochem Biophys Res Commun 141:870-7.
27. Barilla-LaBarca, M. L., Liszewski, M. K., Lambris, J. D., Hourcade, D. & Atkinson, J. P. (2002) Role of membrane cofactor protein (CD46) in regulation of C4b and C3b deposited on cells. J Immunol 168:6298-6304.
28. Fang, C. J., Fremeaux-Bacchi, V., Liszewski, M. K., Pianetti, G., Noris, M., Goodship, T. H. J. & Atkinson, J. P. (2008) Membrane cofactor protein mutations in atypical hemolytic uremic syndrome (aHUS), fatal Stx-HUS, C3 glomerulonephritis and the HEELP syndrome. Blood 111:624-632.
29. Liszewski, M. K., Farries, T. C., Lublin, D. M., Rooney, I. A. & Atkinson, J. P. (1996) Control of the complement system. Adv Immunol 61:201-283.
30. Liszewski, M. K., Leung, M. K., Bertram, P. & Atkinson, J. P. (2007) Dissecting complement regulatory sites of the smallpox inhibitor of complement and abrogating function with a monoclonal antibody. Journal of Immunology 178:44.37.
31. Liszewski, M. K., Post, T. W. & Atkinson, J. P. (1991) Membrane cofactor protein (MCP or CD46): newest member of the regulators of complement activation gene cluster. Annual Review of Immunology 9:431-55.
32. Liszewski, M. K. & Atkinson, J. P. (1996) Membrane cofactor protein (MCP; CD46). Isoforms differ in protection against the classical pathway of complement. J Immunol 156:4415-4421.
33. Liszewski, M. K., Leung, M. K., Schraml, B., Goodship, T. H. & Atkinson, J. P. (2007) Modeling how CD46 deficiency predisposes to atypical hemolytic uremic syndrome. Molecular Immunology 44:1559-1568.
34. Liszewski, M. K., Leung, M. K. & Atkinson, J. P. (1998) Membrane cofactor protein (CD46): importance of N— and O-glycosylation for complement regulatory function. J Immunol 161:3711-3718.
35. Bozzette, S. A., Boer, R., Bhatnagar, V., Brower, J. L., Keeler, E. B., Morton, S. C. & Stoto, M. A. (2003) A model for a smallpox-vaccination policy. N Engl J Med 348:416-25.
36. Friedman, H. M. (2003) Immune evasion by herpes simplex virus type 1, strategies for virus survival. Transactions of the American Clinical and Climatological Association 114:103-112.
37. Rosengard, A. M., Alonso, L. C., Korb, L. C., Baldwin, W. M., 3rd, Sanfilippo, F., Turka, L. A. & Ahearn, J. M. (1999) Functional characterization of soluble and membrane-bound forms of vaccinia virus complement control protein (VCP). Mol Immunol 36:685-97.
38. Ferreira, V. P., Herbert, A. P., Hocking, H. G., Barlow, P. N. & Pangburn, M. K. (2006) Critical role of the C-terminal domains of factor H in regulating complement activation at cell surfaces. J Immunol 177:6308-16.
39. Meri, S. (2007) Loss of self-control in the complement system and innate autoreactivity. Ann NY Acad Sci 1109:93-105.
40. de Cordoba, S. R. & de Jorge, E. G. (2008) Translational mini-review series on complement factor H: genetics and disease associations of human complement factor H. Clin Exp Immunol 151:1-13.
41. Kavanagh, D., Goodship, T. H. & Richards, A. (2006) Atypical haemolytic uraemic syndrome. Br Med Bull 77-78:5-22.
42. Vaziri-Sani, F., Holmberg, L., Sjoholm, A. G., Kristoffersson, A. C., Manea, M., Fremeaux-Bacchi, V., Fehrman-Ekholm, I., Raafat, R. & Karpman, D. (2006) Phenotypic expression of factor H mutations in patients with atypical hemolytic uremic syndrome. Kidney Int 69:981-8.
43. Pickering, M. C. & Cook, H. T. (2008) Translational mini-review series on complement factor H: renal diseases associated with complement factor H: novel insights from humans and animals. Clin Exp Immunol 151:210-30.
44. Noris, M. & Remuzzi, G. (2008) Translational mini-review series on complement factor H: therapies of renal diseases associated with complement factor H abnormalities: atypical haemolytic uraemic syndrome and membranoproliferative glomerulonephritis. Clin Exp Immunol 151:199-209.
45. Schmidt, C. Q., Herbert, A. P., Hocking, H. G., Uhrin, D. & Barlow, P. N. (2008) Translational mini-review series on complement factor H: structural and functional correlations for factor H. Clin Exp Immunol 151:14-24.
46. Prosser, B. E., Johnson, S., Roversi, P., Herbert, A. P., Blaum, B. S., Tyrrell, J., Jowitt, T. A., Clark, S. J., Tarelli, E., Uhrin, D., Barlow, P. N., Sim, R. B., Day, A. J. & Lea, S. M. (2007) Structural basis for complement factor H linked age-related macular degeneration. J Exp Med 204:2277-83.
47. Mullick J, Bernet J, Panse Y, Hallihosur S, Singh A K, Sahu A. Identification of complement regulatory domains in vaccinia virus complement control protein. J Virol 2005;79(19):12382-93.
48. Liszewski M K, Leung M, Cui W, et al. Dissecting sites important for complement regulatory activity in membrane cofactor protein (MCP; CD46). J Biol Chem 2000;275:37692-701.

What is claimed is:

1. An antibody that binds to and substantially inhibits the activity of at least one poxvirus complement inhibitor.

2. The antibody of claim 1, wherein the antibody substantially inhibits the activity of a poxvirus complement inhibitor selected from the group consisting of SPICE, MOPICE, VCP, and EMICE.

3. The antibody of claim 1, wherein the antibody substantially inhibits the activity of at least two poxvirus complement inhibitors.

4. The antibody of claim 1, wherein the antibody substantially inhibits the activity of at least three poxvirus complement inhibitors.

5. The antibody of claim 1, wherein the antibody substantially inhibits the activity of SPICE, MOPICE, VCP, and EMICE.

6. The antibody of claim 1, wherein the antibody substantially binds to and inhibits the activity of SPICE.

7. The antibody of claim 1, wherein the antibody substantially inhibits the cofactor activity of the poxvirus complement inhibitor.

8. The antibody of claim 1, wherein the antibody substantially inhibits the decay accelerating activity of the poxvirus complement inhibitor.

9. The antibody of claim 1, wherein the antibody substantially inhibits the activity of at least one poxvirus complement inhibitor in vivo.

10. The antibody of claim 1, wherein the antibody binds to the dimeric form of the poxvirus complement inhibitor.

11. The antibody of claim 1, wherein the antibody binds to the poxvirus complement inhibitor attached to a virion or a cell.

12. The antibody of claim 1, wherein the antibody binds to the N-terminal complement control protein module.

13. The antibody of claim 1, wherein the antibody binds to the poxvirus complement inhibitor under reducing or non-reducing conditions.

14. The antibody of claim 1, wherein the antibody is labeled.

15. The antibody of claim 1, wherein the label is fluorescent, radioactive, enzymatic, luminescent, or colorimetric.

16. The antibody of claim 1, wherein the antibody is a monoclonal antibody.

17. A method for decreasing the activity of a poxvirus complement inhibitor, the method comprising contacting the poxvirus complement inhibitor with the antibody of claim 1.

18. A method for detecting a poxvirus complement inhibitor in a sample, the method comprising contacting the sample with an antibody of claim 1, and detecting association between the antibody and the poxvirus complement inhibitor.

19. The method of claim 18, wherein the antibody is labeled.

20. The method of claim 19, wherein the label is fluorescent, radioactive, enzymatic, luminescent, or colorimetric.


SEQUENCE LISTING (4 sequences)

SEQ ID NO: 1 - protein, 243 aa, Variola virus
CCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYYLIGEYKSYCKLGSTGSMVWNPKAPICESVKCQLPPSISNGRHNGYNDFYTDGSVVTYSCNSGYSLIGNSGVLCSGGEWSNPPTCQIVKCPHPTILNGYLSSGFKRSYSYNDNVDFTCKYGYKLSGSSSSCSPGNTWQPELPKCVR

SEQ ID NO: 2 - protein, 243 aa, Vaccinia virus
CCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSCSPGNTWKPELPKCVR

SEQ ID NO: 3 - protein, 14 aa, Variola virus
GYYLIGEYKSYCKL

SEQ ID NO: 4 - protein, 14 aa, Homo sapiens
GYYLIGEEILYCEL